(19) United States
(12) Patent Application Publication
(10) Pub. No.: US 2007/0172517 A1
(43) Pub. Date: Jul. 26, 2007

(54) Compositions capable of facilitation penetration across a biological barrier

(76) Inventors: Shmuel A. Ben-Sasson, Jerusalem (IL); Einat Galamidi-Cohen, Jerusalem (IL)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 10/572,249

(22) PCT Filed: Sep. 17, 2004
(86) PCT No.: PCT/IB04/04452

§ 371 Date: Dec. 13, 2006

Related U.S. Application Data
(60) Provisional application No. 60/503,615, filed on Sep. 17, 2003.

(30) Foreign Application Priority Data
Sep. 17, 2003 (US) 10665184
Sep. 17, 2003 (US) 10664989

(51) Int. Cl. A61L 15/16; A61K 38/28; A61K 38/21; A61K 39/395; A61F 13/02; A61K 38/18; A61K 38/19; A61K 38/20; A61K 38/27; A61K 38/29; A61K 38/30; A61K 31/714; A61K 31/7034

(52) U.S. Cl. 424/448; 514/44; 424/85.2; 424/85.1; 514/3; 514/12; 424/145.1; 424/85.6; 424/85.7; 514/52; 514/35; 514/449; 514/15; 514/54; 514/56

(57) ABSTRACT

This invention relates to novel pharmaceutical compositions capable of facilitating penetration of at least one effector across biological barriers. The invention also relates to methods of treating or preventing diseases by administering these compositions to affected subjects.

### TECHNICAL FIELD OF THE INVENTION

This invention relates to novel hydrophobic compositions capable of facilitating penetration of an effector across biological barriers.

### BACKGROUND OF THE INVENTION

Techniques enabling efficient transfer of a substance of interest across a biological barrier are of considerable interest in the field of biotechnology. For example, such techniques may be used for the transport of a variety of different substances across a biological barrier regulated by tight junctions (i.e., the mucosal epithelia, which includes the intestinal and respiratory epithelia and the vascular endothelia, which includes the blood-brain barrier).

The intestinal epithelium represents the major barrier to absorption of orally administered compounds, e.g., drugs and peptides, into the systemic circulation. This barrier is composed of a single layer of columnar epithelial cells (primarily enterocytes, goblet cells, endocrine cells, and paneth cells), which are joined at their apical surfaces by the tight junctions. See Madara et al., PHYSIOLOGY OF THE GASTROINTESTINAL TRACT; 2^{nd} Ed., Johnson, ed., Raven Press, New York, pp. 1251-66 (1987).

Compounds that are presented in the intestinal lumen can enter the blood stream through active or facilitative trasport, passive transcellular transport, or passive paracellular transport. Active or facilitative transport occurs via cellular carriers, and is limited to trasport of low molecular weight degradation products of complex molecules such as proteins and sugars, e.g., amino acids, pentoses, and hexoses. Passive transcellular transport requires partitioning of the molecule through both the apical and basolateral membranes. This process is limited to relatively small hydrophobic compounds. See Jackson, PHYSIOLOGY OF THE GASTROINTESTINAL TRACT; 2nd Ed., Johnson, ed., Raven Press, New York, pp. 1597-1621 (1987). Consequently, with the exception of those molecules that are transported by active or facilitative mechanisms, absorption of larger, more hydrophilic molecules is, for the most part, limited to the paracellular pathway. However, the entry of molecules through the paracellular pathway is primarily restricted by the presence of the tight junctions. See Gumbiner, Am. J. Physiol., 253:C749-C758 (1987); Madara, J. Clin. Invest., 83:1089-94 (1989).

Considerable attention has been directed to finding ways to increase paracellular 15 transport by “loosening” tight junctions. One approach to overcoming the restriction to paracellular transport is to co-administer, in a mixture, biologically active ingredients with absorption enhancing agents. Generally, intestinal/respiratory absorption enhancers include, but are not limited to, calcium chelators, such as citrate and ethylenediamine tetraacetic acid (EDTA) and surfactants, such as sodium dodecyl sulfate, bile salts, palmitoylcarnitine, and sodium salts of fatty acids. For example, EDTA, which is known to disrupt tight junctions by chelating calcium, enhances the efficiency of gene transfer into the airway respiratory epithelium in patients with cystic fibrosis. See Wang, et al., Am. J. Respir. Cell Mol. Biol., 22:129-138 (2000). However, one drawback to all of these methods is that they facilitate the indiscriminate penetration of any nearby molecule that happens to be in the gastrointestinal or airway lumen. In addition, each of these intestinal/respiratory absorption enhancers has properties that limit their general usefulness as a means to promote absorption of various molecules across a biological barrier.

Moreover, with the use of surfactants, the potential lytic nature of these agents raises concerns regarding safety. Specifically, the intestinal and respiratory epithelia provides a barrier to the entry of toxins, bacteria and viruses from the hostile exterior. Hence, the possibility of exfoliation of the epithelium using surfactants, as well as the potential complications arising from increased epithelial repair, raise safety concerns about the use of surfactants as intestinal/respiratory absorption enhancers.

When calcium chelators are used as intestinal/respiratory absorption enhancers, Ca⁺² depletion does not act directly on the tight junction, but, rather, induces global changes in the cells, including disruption of actin filaments, disruption of adherent junctions, diminished cell adhesion, and activation of protein kinases. See Citi, J. Cell Biol., 117:169-178 (1992). Moreover, as typical calcium chelators only have access to the mucosal surface, and luminal Ca⁺² concentration may vary, sufficient amounts of chelators generally cannot be administered to lower Ca⁺² levels to induce the opening of tight junctions in a rapid, reversible, and reproducible manner.

Additionally, some toxins such as Clostridium difficile toxin A and B, appear to irreversibly increase paracellular permeability and are thus, associated with destruction of the tight junction complex. See Hecht, et al., J. Clin. Invest., 82:1516-24 (1988); Fiorentini and Thelestam, Toxicon, 29:543-67 (1991). Other toxins such as Vibrio cholerae zonula occludens toxin (ZOT) modulate the structure of intercellular tight junctions. As a result, the intestinal mucosa becomes more permeable. See Fasano, et al., Proc. Nat. Acad. Sci., USA, 8:5242-46 (1991); U.S. Pat. No. 5,827,534. However, this also results in diarrhea.

Therefore, large hydrophilic molecules of therapeutic value present a difficult problem in the field of drug delivery. While they are readily soluble in water, and thus easily dissolve in physiological media, such molecules are barred from absorption by the mucosal layer due to their cell-membrane impermeability. The epithelial cell membrane is composed of a phospholipid bilayer in which proteins are embedded via hydrophobic segments. Thus, the cell membrane constitutes a very strong barrier for transport of hydrophilic substances, including peptides and proteins.

Several new methods for the delivery of proteins across cell membranes are being evaluated, although these are still lacking in convenience and effectiveness. The most popular method utilizes “protein transduction domains” or “membrane transport signals”. These are derived from viral proteins, or synthetically from phage display libraries, and are characterized by a high content of positively charged lysine and arginine residues. See Schwarze, et al., Science, 285:1569-1572 (1999); Rojas, et al., Nat. Biotechnol., 16:370-375 (1998). Microinjection and electroporation techniques have also been utilized with varying degrees of success.

Lately, alternative methods using a cationic lipid formulation have been suggested. See Zelphati, et al., J. Biol. Chem., 276: 35103-35110, who utilize trifluoroacetylated lipopolyamine and dioleoyl phosphatidylethanolamine, for the delivery of proteins and peptides into the cytoplasm. See also the use of lipoamino acid conjugates and liposaccharide conjugates by Toth, et al., J. Drug Targeting, 2:217-239 (1994), and proceedings thereof. These methods all utilize amphipathic molecules which bind, covalently 6r otherwise, the target molecule, thus “hydrophobizing” its original charge and enabling its penetration through the lipophylic cell membrane.

The use of amphipathic counter ions shows promise for an efficient means for the delivery of therapeutic agents. To date, however, only about 1-3% of the total amount of therapeutic agent administered in conjunction with such counter ions effectively penetrates across the biological barrier.

Thus, a need remains for an efficient, specific, non-invasive, low-risk means for the delivery of biologically active molecules, such as polypeptides, drugs and other therapeutic agents, across various biological barriers.

### SUMMARY OF THE INVENTION

The present invention provides compositions for effectively translocating therapeutically active molecules, i.e., effectors, which are otherwise impermeable to biological barriers, by selectively encapsulating such molecules into a hydrophobic complex. The invention also relates to methods of using a counter ion to the effector to selectively encapsulate and translocate at least one effector across a biological barrier. The counter ion can include a hydrophobic moiety. Specifically, the invention involves a hydrophobic composition sequentially coupled to a therapeutically effective amount of at least one effector, and a counter ion to the at least one effector thereby selectively encapsulating the effector and effectively translocating the effector across a biological barrier. For example, such a compound may be used for transepithelial delivery of at least one effector across a biological barrier. “Effective translocation” as used herein means that introduction of the composition to a biological barrier results in at least 5%, but preferably at least 10%, and even more preferably, at least 20% or more, translocation of the effector across the biological barrier. The at least one effector of the composition is selectively encapsulated in such a way that introduction of the composition to a biological barrier results in translocation of the encapsulated effector only, i.e., no other molecules concomitantly administered in a non-encapsulated or free form are translocated across the biological barrier.

As used herein a “hydrophobic composition” includes any composition that is water insoluble and facilitates the selective encapsulation, or the effective translocation, of a substance, e.g., at least one effector, across a biological barrier utilizing at least one counter ion and at least one pharmaceutically acceptable hydrophobic agent. As used herein, the term “biological barrier” is meant to include biological membranes such as the plasma membrane as well as any biological structures sealed by tight junctions (or occluding junctions) such as the mucosal or vascular epithelia, including, but not limited to, the intestinal or respiratory epithelia, and the blood brain barrier. Moreover, those skilled in the art will recognize that translocation may occur across a biological barrier in a tissue such as epithelial cells or endothelial cells.

As used herein, the term “encapsulation” refers to the introduction of the at least one effector to the hydrophobic composition. The method of encapsulation can involve complex formation of at least one effector with at least one amphipathic counter ion, and dissolution in water or in an at least partially water soluble solvent. The composition can be further supplemented by a protein stabilizer, a penetrating peptide, and/or one or more pharmaceutically acceptable hydrophobic agents. Any one or more of the components of the composition may be lyophilized at various stages of the encapsulation process.

A hydrophobic agent can be a single molecule or a combination of hydrophobic molecules, like aliphatic, cyclic, or aromatic molecules. Examples of aliphatic hydrophobic agents include mineral oil, paraffin, fatty acids, mono-, di-, or tri-glycerides, ethers, or esters. Examples of tri-glycerides include long chain triglycerides, medium chain triglycerides, and short chain triglycerides. Specific examples of suitable triglycerides include tributyrin, trihexanoin, trioctanoin, and tricaprin (1,2,3-tridecanoyl glycerol). Examples of cyclic hydrophobic agents include terpenoids, cholesterol, cholesterol derivatives and cholesterol esters of fatty acids. An example of an aromatic hydrophobic agent includes benzyl benzoate.

At least partially water soluble solvents include, for example, n-butanol, isoamyl (=isopentyl) alcohol, DMF, DMSO, iso-butanol, iso-propanol, propanol, ethanol, ter-butanol, polyols, ethers, amides, esters, or various mixtures thereof.

The invention also provides hydrophobic compositions having a pharmaceutically acceptable carrier or excipient, or a combination thereof. In various embodiments, the compositions of the invention can be contained within a capsule, or can take the form of a tablet, an aqueous dispersion, suspension, or emulsion, a cream, an ointment, a nasal spray, or a suppository. The compositions of the invention can also be enteric-coated.

Hydrophobic compositions can include at least one effector coupled to a suitable counter ion. The at least one effector can be a therapeutically active cationic or anionic impermeable molecule including, but not limited to, nucleic acids; glycosaminoglycans; proteins; peptides; or pharmaceutically active agents, such as, for example, hormones, growth factors, neurotrophic factors, anticoagulants, bioactive molecules, toxins, antibiotics, anti-fungal agents, antipathogenic agents, antigens, antibodies, antibody fragments, immunomodulators, vitamins, antineoplastic agents, enzymes, or therapeutic agents. For example, glycosaminoglycans acting as anionic impermeable compounds include, but are not limited to, heparin, heparan sulfate, chondroitin sulfate, dermatan sulfate, and hyaluronic acid. Nucleic acids serving as anionic impermeable molecules include, but are not limited to, specific DNA sequences (e.g., coding genes), specific RNA sequences (e.g., RNA aptamers, antisense RNA or a specific inhibitory RNA (RNAi)), poly CpG, or poly I:C synthetic polymers of nucleic acids. Suitable pharmaceutically active agents also include vitamin B12, taxol, Caspofungin, or an aminoglycoside antibiotic (e.g. Gentamycin, Amikacin, Tobramycin, or Neomycin). Other suitable proteins include, but are not limited to, hormones, gonadotropins, growth factors, cytokines, neurotrophic factors, immunomodulators, enzymes, anticoagulants, toxins, antigens, antipathogenic agents, antineoplastic agents, antibodies, antibody fragments, and other therapeutic agents. Specifically these include, but are not limited to, insulin, erythropoietin (EPO), glucagon-like peptide 1 (GLP-1), αMSH, parathyroid hormone (PTH), growth hormone, calcitonin, interleukin-2 (IL-2), α1-antitrypsin, granulocyte/monocyte colony stimulating factor (GM-CSF), granulocyte colony stimulating factor (G-CSF), T20, anti-TNF antibodies, interferon α, interferon β, interferon γ, lutenizing hormone (LH), follicle-stimulating hormone (FSH), enkephalin, dalargin, kyotorphin, basic fibroblast growth factor (bFGF), hirudin, hirulog, lutenizing hormone releasing hormone (LHRH) analog, brain-derived natriuretic peptide (BNP), glatiramer acetate (Copolymer-1), and neurotrophic factors.

In any of the compositions of the invention, the effector may additionally contain at least one chemical modification. For example, the chemical modification may be the attachment of one or more polyethylene glycol residues to the effector. Additionally, any of the compostions of the invention may also further contain water or an at least partially water soluble solvent selected from the group consisting of n-butanol, isoamyl (=isopentyl) alcohol, DMF, DMSO, iso-butanol, iso-propanol, propanol, ethanol, ter-butanol, polyols, ethers, amides, esters, and various mixtures thereof.

As used herein, “cationic or anionic impermeable molecules” are molecules that are positively (cationic) or negatively (anionic) charged and are unable to efficiently cross biological barriers, such as the cell membrane or tight junctions. Preferably, cationic and anionic impermeable molecules of the invention are of a molecular weight above 200 Daltons. Anionic impermeable molecules are preferably polysaccharides, i.e., glycosaminoglycans, nucleic acids, or net negatively charged proteins, whereas cationic impermeable molecules are preferably net positively charged proteins. A protein's net charge is determined by two factors: 1) the total count of acidic amino acids vs. basic amino acids, and 2) the specific solvent pH surroundings, which expose positive or negative residues. As used herein, “net positively or net negatively charged proteins” are proteins that, under non-denaturing pH surroundings, have a net positive or net negative electric charge. For example, interferon β is a protein that contains 23 positively charged residues (lysines and arginines), and 18 negatively charged residues (glutamic or aspartic acid residues). Therefore, under neutral or acidic pH surroundings, interferon β constitutes a net positively charged protein. Conversely, insulin is a 51 amino acid protein that contains two positively charged residues, one lysine and one arginine, and four glutamic acid residues. Therefore, under neutral or basic pH surroundings, insulin constitutes a net negatively charged protein. In general, those skilled in the art will recognize that all proteins may be considered “net negatively charged proteins” or “net positively charged proteins”, regardless of their amino acid composition, depending on their pH and/or solvent surroundings. For example, different solvents can expose negative or positive side chains depending on the solvent pH.

Compositions according to the invention can also be used to enhance the penetration of smaller molecules that are otherwise impermeable through epithelial barriers. Examples of such molecules include nucleic acids (ie., DNA, RNA, or mirnetics thereof), where the counter ion is cationic. Conversely, when the counter ion is anionic, molecules such as Caspofungin, vitamin B12, and aminoglycoside antibiotics (e.g. Gentamycin, Amikacin, Tobramycin, or Neomycin) can penetrate through epithelial barriers.

Counter ions of this invention can include, for example, anionic or cationic amphipathic molecules. In one embodiment, anionic or cationic counter ions of this invention are ions that are negatively (anionic) or positively (cationic) charged and can include a hydrophobic moiety. Under appropriate conditions, anionic or cationic counter ions can establish electrostatic interactions with cationic or anionic impermeable molecules, respectively. The formation of such a complex can cause charge neutralization, thereby creating a new uncharged entity, with further hydrophobic properties in the case of an inherent hydrophobicity of the counter ion.

For example, suitable anionic amphipathic molecules may include an organic acid such as carboxylate, sulfonate, and phosphonate anion, wherein the amphipathic molecule comprises a hydrophobic moiety. Specifically, the counter ion may be sodium dodecyl sulphate or dioctyl sulfosuccinate.

Contemplated cationic counter ions include quaternary amine derivatives, such as benzalkonium derivatives. Suitable quaternary amines can be substituted by hydrophobic residues. In general, quaternary amines contemplated by the invention have the structure: 1-R1-2-R2-3-R3-4-R4-N, wherein R1,2,3, and 4 are alkyl or aryl derivatives. For example, the quaternary amine may be a benzalkonium derivative. 30 Further, quaternary amines can be ionic liquid forming cations, such as imidazolium derivatives, pyridinium derivatives, phosphonium compounds or tetralkylammonium compounds. Ionic liquid forming cations may be constituents of a water soluble salt. For example, imidazolium derivatives have the general structure of 1-R1-3-R2-imidazolium where R1 and R2 can be linear or branched alkyls with 1 to 12 carbons. Such imidazolium derivatives can be further substituted for example by halogens or an alkyl group. Specific imidazolium derivatives include, but are not limited to, 1-ethyl-3-methylimidazolium, 1-butyl-3-methylimidazolium, 1-hexyl-3-methylimidazolium, 1-methyl-3-octylimidazolium, 1-methyl-3-(3,3,4,4,5,5,6,6,7,7,8,8,8-tridecafluoroctyl)-imidazolium, 1,3-dimethylimidazolium, and 1,2-dimethyl-3-propylimidazolium.

Pyridinium derivatives have the general structure of 1-R1-3-R2-pyridinium where R1 is a linear or branched alkyl with 1 to 12 carbons, and R2 is H or a linear or branched alkyl with 1 to 12 carbons. Such pyridinium derivatives can be further substituted, for example by halogens or an alkyl group. Pyridinium derivatives include, but are not limited to, 3-methyl-1-propylpyridinium, 1-butyl-3-methylpyridinium, and 1-butyl-4-methylpyridinium.

The invention also involves methods of selectively encapsulating and effectively translocating at least one effector across a biological barrier using the compositions of the invention. For example, at least one effector can be coupled to a counter ion to form a composition according to the invention, which can then be introduced to a biological barrier, thereby effectively translocating the effector across the biological membrane. The counter ion can further include a hydrophobic moiety. As used herein, the term “coupled” is meant to include all such specific interactions that result in two or more molecules showing a preference for one another relative to some third molecule, including any type of interaction enabling a physical association between an effector and an ionic liquid forming cation. Preferably this includes, but is not limited to, electrostatic interactions, hydrophobic interactions and hydrogen bonding, but does not include non-specific associations such as solvent preferences. The association must be sufficiently strong so that the effector does not dissociate before or during penetration of the biological barrier.

In some embodiments, the invention provides methods for translocating at least one effector across a biological barrier comprising introducing any of the hydrophobic compositions described hereinto a biological barrier and allowing the at least one effector to translocate across said biological barrier. For example, the biological barrier may be located within epithelial cells and endothelial cells. Examples biological barriers contemplated by the invention include tight junctions and/or plasma membranes, such as the gastro-intestinal mucosa and the blood brain barrier.

Any of the hydrophobic compositions of this invention may further contain a penetrating peptide. The penetrating peptides used in compositions of the invention can have at least one amino acid sequence selected from: (BX)₄Z(BX)₂ZXB (SEQ ID NO:44); ZBXB₂XBXB₂XBX₃BXB₂X₂B₂ (SEQ ID NO:45); ZBZX₂B₄XB₃ZXB₄Z₂B₂ SEQ ID NO:46); ZB₉XBX₂B₂ZBXZBX₂ (SEQ ID NO:47); BZB₈XB₉X₂ZXB (SEQ ID NO:48); B₂ZXZB₅XB₂XB₂X₂BZXB₂ (SEQ ID NO:49); XB₉XBXB₆X₃B (SEQ ID NO:50); X₂B₃XB₄ZBXB₄XBₙXB (SEQ ID NO:51); XB₂XZBXZB₂ZXBX₃BZXBX₃B (SEQ ID NO:52); BZXBXZX₂B₄XBX₂B₂XB₄X₂ (SEQ ID NO:53); BZXBXZX₂B₄XBX₂B₂XB₄ (SEQ ID NO:54); B₂XZ₂XB₄XBX₂B₅X₂B₂ (SEQ ID NO:55); B_{q}XₜZBₘX_{q}B₄XBXₙBₘZB₂X₂B₂ (SEQ ID NO:56); B₂ZX₃ZBₘX_{q}B₄XBXₙBₘZB₂X₂B₂(SEQ ID NO:57); X₃ZB₆XBX₃BZB₂X₂B₂ (SEQ ID NO:58); and at least 12 contiguous amino acids of any of these amino acid sequences, where X is any amino acid; B is a hydrophobic amino acid; and Z is a charged amino acid; and where q is 0 or 1; m is 1 or 2; and n is 2 or 3; and where t is 1 or 2 or 3; and where the penetrating peptide is capable of translocating across a biological barrier.

Specifically, the penetrating peptide can have an amino acid sequence of any one of SEQ ID NOS: 1-15 and 24-29. The invention also provides a penetrating peptide having an amino acid sequence of any one of SEQ ID NOS: 22, and 30-37. In addition, the penetrating peptides of the invention include peptides having at least 12 contiguous amino acids of any of the peptides defined by SEQ ID NOS:1-15, 22, and 24-37. The penetrating peptides can be less than thirty (30), less than twenty-five (25), or less than twenty (20) amino acids in length. The invention also includes mutant or variant peptides any of whose residues may be changed from the corresponding residues shown in SEQ ID NOS: 1-15, 22, and 24-37, while still encoding a peptide that maintains its penetrating activities and physiological functions, or functional fragments thereof. For example, the fragment of an amino acid sequence of any one of SEQ ID NOS: 1-15, 22 and 24-37 is at least 10 amino acids in length, and may contain conservative or non-conservative amino acid substitutions.

In general, a penetrating peptide variant that preserves the translocating function includes any variant in which residues at a particular position in the sequence have been substituted by other amino acids, and further includes the possibility of inserting an additional residue or residues between two residues of the parent protein as well as the possibility of deleting one or more residues from the parent sequence. Any such amino acid substitution, insertion, or deletion is encompassed by the invention. In favorable circumstances, the substitution is a conservative substitution.

Amino acid substitutions at “non-essential” amino acid residues can be made in the penetrating peptides. A “non-essential” amino acid residue is a residue that can be altered from the native sequences of the penetrating peptides without altering their biological activity, whereas an “essential” amino acid residue is required for such biological activity. For example, amino acid residues that are conserved among the penetrating peptides of the invention are predicted to be particularly non-amenable to substantial alteration. Amino acids for which conservative substitutions can be made are well known within the art.

Mutations can be introduced into nucleic acids encoding penetrating peptides by standard techniques, including, but not limited to site-directed mutagenesis and PCR-mediated mutagenesis. Preferably, conservative amino acid substitutions are made at one or more predicted, non-essential amino acid residues. A “conservative amino acid substitution” is one in which the amino acid residue is replaced with an amino acid residue having a similar side chain. Families of amino acid residues having similar side chains have been defined within the art These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted non-essential amino acid residue in the penetrating peptide is replaced with another amino acid residue from the same side chain family.

Altematively, mutations can be introduced randomly along all or part of a penetrating peptide coding sequence, such as by saturation mutagenesis, and the resultant mutants can be screened for biological activity to identify mutants that retain activity. Following mutagenesis, the encoded penetrating peptide can be expressed by any recombinant technology known in the art and the activity of the protein can be determined. Amino acid substitutions can also be introduced during artificial peptide synthesis such as solid-phase synthesis of peptides.

The relatedness of amino acid families may also be determined based on side chain interactions. Substituted amino acids may be fully conserved “strong” residues or fully conserved “weak” residues. The “strong” group of conserved amino acid residues may be any one of the following groups: STA, NREQK (SEQ ID NO:17), NHQK (SEQ ID NO:18), NDEQ (SEQ ID NO:19), QHRK (SEQ ID NO:20), MILV (SEQ ID NO:21), MILF (SEQ ID NO:23), HY, FYW, wherein the single letter amino acid codes are grouped by those amino acids that may be substituted for each other. Likewise, the “weak” group of conserved residues may be any one of the following: CSA, ATV, SAG, STNK (SEQ ID NO:38), STPA (SEQ ID NO:39), SGND (SEQ ID NO:40), SNDEQK (SEQ ID NO:41), NDEQHK (SEQ ID NO:42), NEQHRK (SEQ ID NO:43), HFY, wherein the letters within each group represent the single letter amino acid code.

The penetrating peptides of the invention may contain less than 30 amino acids, preferably less than 25 amino acids, most preferably less than 20 amino acids.

The penetrating peptides utilized herein are preferably modified by hydrophobic moieties. The penetrating peptides are then incorporated into the construct of the composition, including the desired effector. The hydrophobization of the penetrating peptide can be achieved via acylation of free amino group(s) of extra lysine(s), interspaced by glycine, alanine, or serine residues, added at the C-terminus of the penetrating peptide. The free amino groups of these lysine residues may be acylated. Acylation of the penetrating peptide preferably utilizes long-chain fatty acids such as stearoyl, palmitoyl, oleyl, ricinoleyl, or myristoyl.

The penetrating peptides of the invention may be further modified via one or more peptidic bonds, to enable protection from gastro-intestinal proteolysis. For example, one or more amino acid residues may be replaced by a non-naturally occurring amino acid such as D-amino acids, norleucine, norvaline, homocysteine, homoserine, ethionine, and compounds derivatized with an amino-terminal blocking group selected from the group consisting of t-butyloxycarbonyl, acetyl, methyl, succinyl, methoxysuccinyl, suberyl, adipyl, azelayl, dansyl, benzyloxycarbonyl, fluorenylmethoxycarbonyl, methoxyaselayl, methoxyadipyl, methoxysuberyl, and a 2,3-dinitrophenyl group. Likewise, one or more peptide bonds may be replaced with an alternative type of covalent bond to form a peptide mimetic.

The penetrating peptides of the invention can also include amino acid analogs in which one or more peptide bonds have been replaced with an alternative type of covalent bond (a “peptide mimetic”) that is not susceptible to cleavage by peptidases elaborated by the subject. Where proteolytic degradation of a peptide composition is encountered following administration to the subject, replacement of one or more particularly sensitive peptide bonds with a noncleavable peptide mimetic renders the resulting peptide derivative compound more stable, and thus, more useful as a therapeutic. Such mimetics, and methods of incorporating them into peptides, are well known in the art.

Similarly, the replacement of an L-amino acid residue by a D-amino acid residue is one standard method for rendering the compound less sensitive to enzymatic destruction. Other amino acid analogs are known in the art, such as norleucine, norvaline, homocysteine, homoserine, ethionine, and the like. Also useful is derivatizing the compound with an amino-terminal blocking group such as a t-butyloxycarbonyl, acetyl, methyl, succinyl, methoxysuccinyl, suberyl, adipyl, azelayl, dansyl, benzyloxycarbonyl, fluorenylmethoxycarbonyl, methoxyaselayl, methoxyadipyl, methoxysuberyl, and a 2,3-dinitrophenyl group.

The penetrating peptides of the invention can also be synthesized using solid-phase synthesis.

The penetrating peptides of the invention can also be further chemically modified. For example, one or more polyethylene glycol (PEG) residues can be attached to the penetrating peptides of the invention.

The invention also includes penetrating peptides that are derived from a bacterial protein. In one embodiment, the invention provides a penetrating peptide derived from a bacterial protein having an amino acid sequence of any one of SEQ ID NOS:1-8, 10-15 and 25-29. Such a penetrating peptide can be derived from an integral membrane protein, a bacterial toxin, or an extracellular protein. The penetrating peptide can also be derived from a human neurokinin receptor. In another embodiment, the invention provides a peptide derived from a neurokinin receptor having an amino acid sequence of any one of SEQ ID NOS:9 and 24.

The compositions of the invention involve the coupling of the penetrating peptide to the effector. As defined above, the term “coupled” is meant to include all such specific interactions that result in two or more molecules showing a preference for one another relative to some third molecule, including any type of interaction enabling a physical association between an effector and a penetrating peptide. Preferably, this includes, but is not limited to, electrostatic interactions, hydrophobic interactions and hydrogen bonding, but does not include non-specific associations such as solvent preferences. The association must be sufficiently strong so that the effector does not dissociate before or during penetration of the biological barrier.

Furthermore, the coupling of the effector to the penetrating peptide can also be achieved indirectly via a mediator. For example, such a mediator can be a large hydrophobic molecule, such as a triglyceride, that binds the effector-counter ion complex, on the one hand, and the hydrophobized penetrating peptide, on the other hand.

The invention also includes methods of producing compositions of the invention by coupling a therapeutically effective amount of at least one effector with a penetrating peptide and a counter-ion to the effector. Such coupling can be via a non-covalent bond. The non-covalent bond can be achieved by adding a hydrophobic moiety to the penetrating peptide, such that the moiety enables the penetrating peptide to be incorporated at the interface of the hydrophobic vesicle in which the effector is contained.

The hydrophobic compositions of this invention may further contain a stabilizer of protein structure. “Stabilizers of protein structure” or “protein stabilizers”, as used herein, refer to any compounds that can stabilize protein structure under aqueous or non-aqueous conditions. Such protein stabilizers include polycationic molecules, polyanionic molecules, and uncharged polymers. One example of a polycationic molecule that can function as a protein stabilizer is a polyamine such as spermine. Examples of polyanionic molecules that can function as protein stabilizers include phytic acid and sucrose octasulfate. Examples of uncharged polymers that can function as protein stabilizers include polyvinylpyrrolidone and polyvinyl alcohol.

The hydrophobic compositions of this invention may also contain a surface active agent. Suitable surface active agents include ionic and non-ionic detergents. Ionic detergents can be fatty acid salts, lecithin, or bile salts. Examples of non-ionic detergents include cremophore, a polyethylene glycol fatty alcohol ether, Solutol HS15, sorbitan fatty acid esters, or a poloxamer. Examples of sorbitan fatty acid esters include sorbitan monolaurate, sorbitan monooleate, and sorbitan monopalmitate.

The hydrophobic compositions of this invention can further contain a protective agent. An example of a protective agent is a protease inhibitor. Suitable protease inhibitors that can be added to the composition are described in Bermkop-Schnurch et al., J. Control. Release, 52:1-16 (1998), incorporated herein by reference. These include, for example, inhibitors of luminally secreted proteases, such as aprotinin, Bowman-Birk inhibitor, soybean trypsin inhibitor, chicken ovomucoid, chicken ovoinhibitor, human pancreatic trypsin inhibitor, camostate mesilate, flavonoid inhibitors, antipain, leupeptin,p-aminobenzamidine, AEBSF, TLCK, APMSF, DFP, PMSF, poly(acrylate) derivatives, chymostatin, benzyloxycarbonyl-Pro-Phe-CHO, FK-448, sugar biphenylboronic acids complexes, β-phenylpropionate, elastatinal, methoxysuccinyl-Ala-Ala-Pro-Val-chloromethylketone (MPCMK), EDTA, and chitosan-EDTA conjugates. Suitable protease inhibitors also include inhibitors of membrane bound proteases, such as amino acids, di- and tripeptides, amastatin, bestatin, puromycin, bacitracin, phosphinic acid dipeptide analogues, α-aminoboronic acid derivatives, Na-glycocholate, 1,10-phenantroline, acivicin, L-serine-borate, thiorphan, and phosphoramidon.

Preferred compositions include, e.g., enteric-coated tablets and gelatin capsules comprising the active ingredient together with a) diluents, e.g., lactose, dextrose, sucrose, mannitol, sorbitol, cellulose and/or glycine; b) protease inhibitors such as Aprotinin or trasylol; c) lubricants, e.g., silica, talcum, stearic acid, its magnesium or calcium salt, poloxamer and/or polyethyleneglycol; for tablets also d) binders, e.g., magnesium aluminum silicate, starch paste, gelatin, tragacanth, methylcellulose, sodium carboxymethylcellulose and/or polyvinylpyrrolidone; e) ionic surface active agents such as poloxamer, Solutol HS15, Cremophore, and bile acids, if desired f) disintegrants, e.g., starches, agar, alginic acid or its sodium salt, or effervescent mixtures; and/or g) absorbents, colorants, flavors and sweeteners. Suppositories are advantageously prepared from fatty emulsions or suspensions. The compositions may be sterilized and/or contain adjuvants, such as preserving, reducing agents e.g. NAC (N-Acetyl-L-Cysteine), antioxidants, stabilizing, wetting or emulsifying agents, solution promoters, salts for regulating the osmotic pressure and/or buffers. In addition, they may also contain other therapeutically valuable substances. The compositions are prepared according to conventional mixing, granulating or coating methods, respectively, and contain about 0.001 to 75%, preferably about 0.01 to 10%, of the active ingredient

The compositions may further contain a mixture of at least two substances selected from the group consisting of a non-ionic detergent, an ionic detergent, a protease inhibitor, a sulfohydryl group status modifying agent, and an antioxidant. For example, the non-ionic detergent may be a poloxamer, cremophore, a polyethylene glycol fatty alcohol ether, or Solutol HS15; the ionic detergent may be a fatty acid salt; the protease inhibitor may be selected from the group consisting of aprotinin, Bowman-Birk inhibitor, soybean trypsin inhibitor, chicken ovomucoid, chicken ovoinhibitor, human pancreatic trypsin inhibitor, camostate mesilate, flavonoid inhibitors, antipain, leupeptin, p-aminobenzamidine, AEBSF, TLCK, APMSF, DFP, PMSF, poly(acrylate) derivatives, chymostatin, benzyloxycarbonyl-Pro-Phe-CHO, FK448, sugar biphenylboronic acids complexes, β-phenylpropionate, elastatinal, methoxysuccinyl-Ala-Ala-Pro-Val-chloromethylketone (MPCMK), EDTA, chitosan-EDTA conjugates, amino acids, di-peptides, tripeptides, amastatin, bestatin, puromycin, bacitracin, phosphinic acid dipeptide analogues, α-aminoboronic acid derivatives, Na-glycocholate, 1,10-phenantroline, acivicin, L-serine-borate, thiorphan, phosphoramidon, and combinations thereof; the sulfohydryl group status modifying agent may be N-acetyl cysteine (NAC) or Diamide or combinations thereof; and/or the antioxidant may be selected from the group consisting of tocopherol, deteroxime mesylate, methyl paraben, ethyl paraben, and ascorbic acid and combinations thereof.

The invention also provides kits having one or more containers containing a therapeutically or prophylactically effective amount of a composition of the invention.

Also described are methods of treating or preventing a disease or pathological condition by administering to a subject in which such treatment or prevention is desired, a composition of the invention in an amount sufficient to treat or prevent the disease or pathological condition. For example, the disease or condition to be treated may include but are not limited to endocrine disorders, including diabetes, infertility, hormone deficiencies and osteoporosis; ophthalmological disorders; neurodegenerative disorders, including Alzheimer's disease and other forms of dementia, Parkinson's disease, multiple sclerosis, and Huntington's disease; cardiovascular disorders,; including atherosclerosis, hyper- and hypocoagulable states, coronary disease, and cerebrovascular events; metabolic disorders, including obesity and vitamin deficiencies; renal disorders, including renal failure; haematological disorders, including anemia of different entities; immunologic and rheumatologic disorders, including autoimmune diseases, and immune deficiencies; infectious diseases, including viral, bacterial, fungal and parasitic infections; neoplastic diseases; and multi-factorial disorders, including impotence, chronic pain, depression, different fibrosis states, and short stature.

Administration of the active compounds and salts described herein can be via any of the accepted modes of administration for therapeutic agents. These methods include oral, buccal, anal, rectal, bronchial, nasal, sublingual, parenteral, transdermal, pulmonary, intraorbital, parenteral or topical administration modes.

Also included in the invention are methods of producing the compositions described herein. For example, the effector and the counter ion can be lyophilized or freeze dried together and then reconstituted under preferred solvent surroundings. Any one or more of the protein stabilizers, the penetrating peptides, and/or any other constituent of the pharmaceutical excipient or carrier can be optionally added with the effector and counter ion during the lyophilization. Other components of the composition can also be optionally added during reconstitution of the lyophilized materials. Such optional components can include, for example, pluronic F-68, Aprotinin, Solutol HS15 and/or N-Acetyl Cysteine.

Also provided are methods of mucosal, ie., oral, nasal, rectal, vaginal, or bronchial, vaccination involving administering to a subject in need of vaccination an effective amount of a composition of the invention, wherein the effector includes an antigen to which vaccination is desired. In one embodiment, the effector can be a protective antigen (PA) for use in a vaccine against Anthrax. In another embodiment, the effector can be a Hepatitis B surface antigen (HBs) for use in a vaccine against Hepatitis B.

The details of one or more embodiments of the invention have been set forth in the accompanying description below. Although any methods and materials similar or equivalent to those described herein can be used in the practice or testing of the present invention, the preferred methods and materials are now described. Other features, objects, and advantages of the invention will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this invention belongs. All patents and publications cited in this specification are incorporated by reference.

### BRIEF DESCRIPTION OF THE DRAWINGS

FIG. 1 shows an amino acid sequence alignment of ORF HI0638 and its homologues from other pathogenic bacteria.

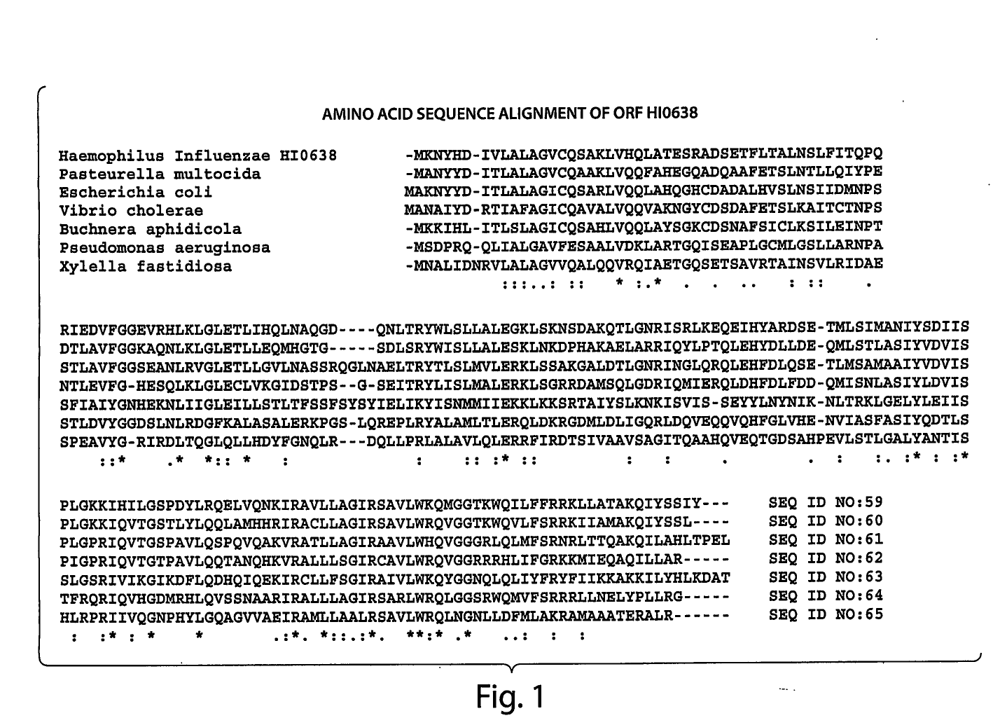

FIG. 2 shows an amino acid sequence alignment of penetrating peptides used in this invention, as well as their organism of origin.

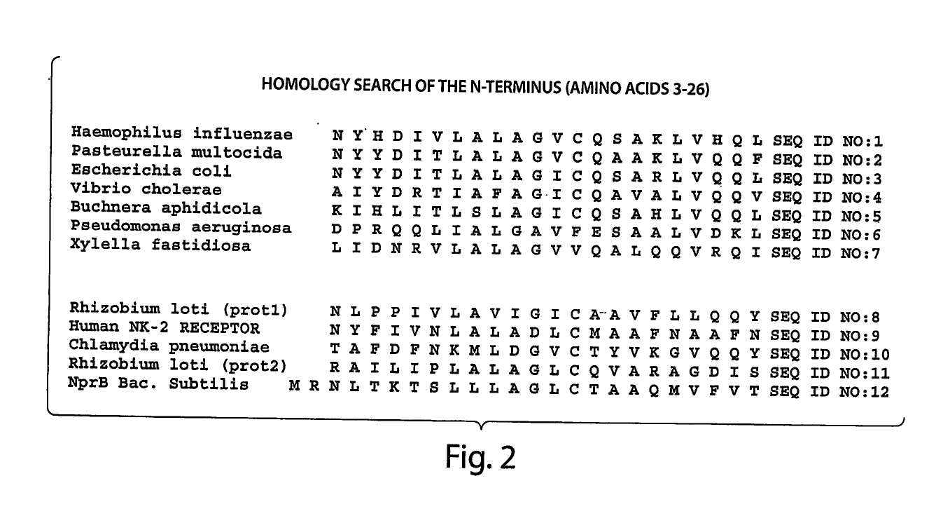

FIG. 3 shows a graph of blood glucose levels in mice plotted against time, following insulin translocation across epithelial cell membranes via administration of the compositions of the invention.

FIG. 4 shows a graph of blood glucose levels in rats plotted against time, following insulin translocation across epithelial cell membranes via administration of the compositions of the invention.

### DETAILED DESCRIPTION OF THE INVENTION

As described herein, cationic or anionic counter ions of the invention can be utilized for enabling or facilitating effective translocation of at least one effector across biological barriers via selective encapsulation. Cationic counter ions of this invention are ions that are positively charged and, in addition, that may include a hydrophobic moiety. Anionic counter ions of this invention are ions that are negatively charged and, in addition, that may include a hydrophobic moiety. Under appropriate conditions, cationic or anionic counter ions can establish electrostatic interactions with anionic or cationic impermeable molecules, respectively. The formation of such a complex can cause charge neutralization, thereby creating a new uncharged entity, with further hydrophobic properties in case of an inherent hydrophobicity of the counter ion.

The use of the effector—counter ion hydrophobic compositions described herein allows for low immunogenicity, high reproducibility, extensive and simple application for a wide variety of therapeutic molecules, and allows for the potential for highly efficient delivery through biological barriers in an organism. Accordingly, these compositions have the potential to improve upon conventional transporters such as liposomes or viruses for the efficient delivery of many macromolecules. The methods of the present invention employ the use of an effector—counter ion complex to create hydrophobic compositions to specifically transport macromolecules across biological barriers that are sealed by tight junctions.

The present invention provides compositions for penetration that specifically target various tissues, especially epithelial and endothelial, for the delivery of drugs and other therapeutic agents across a biological barrier. Existing transport systems known in the art are too limited to be of general application because they are inefficient, they alter the biological properties of the active substance, they kill the target cell, they irreversibly destroy the biological barrier, and/or they pose too high of a risk to be used in human subjects.

In one embodiment, the compositions of the invention contain an impermeable effector and an appropriate counter ion to the effector. This complex can then by lyophilized and reconstituted in a certain order of steps as further described herein, such that a self-assembly of hydrophilic and hydrophobic molecules are produced, whereby the once impermeable effector, and only the effector, is efficiently translocated across a biological barrier. The compositions of the instant invention can be defined by its efficiency, as they must enable translocation of at least 5% (but preferably at least 10% or at least 20%) of the effector across an epithelial barrier. This efficiency is greater than that of other compositions known in the art which typically enable translocation of only about 1-3% of the effector.

The compositions of the present invention exhibit efficient, non-invasive delivery of an unaltered biologically active substance (i.e., an effector), by utilizing selective encapsulation, and thus, have many uses. For example, the compositions of the invention can be used in the treatment of diabetes. Insulin levels in the blood stream must be tightly regulated. The compositions of the invention can be used to deliver insulin, for example, across the mucosal epithelia, at a high yield. Alternative non-invasive insulin delivery methods previously known in the art, have typical yields of 1-4% and cause intolerable fluctuations in the amount of insulin absorbed. Another treatment for elevated blood glucose levels involves the use of glucagon-like peptide 1 (GLP-1). GLP-1 is a potent hormone, which is endogenously secreted in the gastrointestinal tract upon food injection. GLP-1's important physiological action is to augment the secretion of insulin in a glucose-dependant manner, thus allowing for treatment of diabetic states.

In addition, these compositions also can be used to treat conditions resulting from atherosclerosis and the formation of thrombi and emboli such as myocardial infarction and cerebrovascular accidents. Specifically, the compositions can be used to deliver heparin across the mucosal epithelia. Heparin is an established effective and safe anticoagulant. However, its therapeutic use is limited by the need for parenteral adinistration. Thus far, there has been limited success in the direction of increasing heparin absorption from the intestines, and a sustained systemic anticoagulant effect has not been achieved.

The compositions of this invention can also be used to treat hematological diseases and deficiency states that are amenable to administration of hematological growth factors. For example, erythropoietin is a glycoprotein that stimulates red blood cell production. It is produced in the kidney and stimulates the division and differentiation of committed erythroid progenitors in the bone marrow. Endogenously, hypoxia and anemia generally increase the production of erythropoietin, which in turn stimulates erythropoiesis. However, in patients with chronic renal failure (CRF), production of erythropoietin is impaired. This erythropoietin deficiency is the primary cause of their anemia. Recombinant EPO stimulates erythropoiesis in anemic patients with CRF, including patients on dialysis, as well as those who do not require regular dialysis. Additional anemia states treated by EPO include Zidovudine-treated HIV-infected patients, and cancer patients on chemotherapy. Anemia observed in cancer patients may be related to the disease itself or the effect of concomitantly administered chemotherapeutic agents.

Another widespread cause of anemia is pernicious anemia, caused by a lack of vitamin B12. The complex mechanism of vitamin B12 absorption in the gastrointestinal tract involves the secretion and binding to Intrinsic Factor. This process is abnormal in pernicious anemia patients, thereby resulting in lack of vitamin B12 absorption and anemia. The hydrophobic compositions of the invention can be used to deliver vitamin B12 across the mucosal epithelia at high yield.

Colony stimulating factors are glycoproteins which act on hematopoietic cells by binding to specific cell surface receptors and stimulating proliferation, differentiation, commitment, and some end-cell functional activation. Granulocyte-colony stimulation factor (G-CSF) regulates the production of neutrophils within the bone marrow and affects neutrophil progenitor proliferation, differentiation and selected end-cell functional activation, including enhanced phagocytic ability, priming of the cellular metabolism associated with respiratory burst, antibody dependent killing, and the increased expression of some functions associated with cell surface antigens.

In cancer patients, recombinant granulocyte-colony stimulating factor has been shown to be safe and effective in accelerating the recovery of neutrophil counts following a variety of chemotherapy regimens, thus preventing hazardous infectious. G-CSF can also shorten bone marrow recovery when administered after bone marrow trasplantations.

The composition of this invention can also be used to administer monoclonal antibodies for different indications. For example, administration of antibodies that block the signal of tumor necrosis factor (TNF) can be used to treat pathologic inflammatory processes such as rheumatoid arthritis (RA), polyarticular-course juvenile rheumatoid arthritis (JRA), as well as the resulting joint pathology.

Additionally, the compositions of this invention can also be used to treat osteoporosis. It has recently been shown that intermittent exposure to parathyroid hormone (PTH), as occurs in recombinant PTH injections, results in an anabolic response, rather than the well known catabolic reaction induced by sustained exposure to elevated PTH levels, as seen in hyperparathyroidism. Thus, non invasive administration of PTH may be beneficial for increasing bone mass in various deficiency states, including osteoporosis. See Fox, Curr. Opin. Pharmacol., 2:338-344 (2002).

The compositions of the invention can also be used in the treatment of bacterial infections. Since the introduction of penicillins, pathogenic bacteria have been steadily acquiring novel mechanisms enabling a growing resistance to antibiotic therapy. The expanding number of highly insensitive bacterial pathogens presents an ever-growing challenge to physicians and caregivers. Consequently, patients are often forced to remain hospitalized for long periods, in order to receive IV antibiotic therapy, with obvious economic and medical disadvantages. Aminoglycoside antibiotics are potent antibacterial antibiotics that are ineffectively absorbed through biological barriers. Thus, the compositions of the invention can be used to deliver aminoglycosides, such as gentamycin, tobramycin, neomycin, and amikacin, across the mucosal epithelia at high yield.

Currently, the delivery of effectors (e.g., the delivery of gentamycin, insulin, heparin, or erythropoietin to the blood stream (or the like)) requires invasive techniques such as intravenous or intramuscular injections. One advantage of the compositions of the invention is that they can deliver effectors across biological barriers through non-invasive means of administration, including, for example oral, nasal, bucal, rectal, inhalation, insufflation, transdermal, or depository. In addition, a further advantage of the compositions of the invention is that they are able to cross the blood-brain barrier, thereby delivering effectors to the central nervous system (CNS).

Compositions of the invention facilitate the passage, translocation, or penetration of a substance across a biological barrier, particularly through or between cells “sealed” by tight junctions. Translocation may be detected by any method known to those skilled in the art, including using imaging compounds, such as radioactive tagging, and/or fluorescent probes or dyes, incorporated into a hydrophobic composition in conjunction with a paracytosis assay as described in, for example, Schilfgaarde, et al., Infect. and Immun., 68(8):4616-23 (2000). Generally, a paracytosis assay is performed by: a) incubating a cell layer with a hydrophobic composition described by this invention; b) making cross sections of the cell layers; and c) detecting the presence of a component of the compositions of the invention such as effectors, counter ions, or penetrating peptides. The detection step may be carried out by incubating the fixed cell sections with labeled antibodies directed to a component of the compositions of this invention, followed by detection of an immunological reaction between the component and the labeled antibody. Alternatively, a component may be labeled using a radioactive label, or a fluorescent label, or a dye in order to directly detect the presence of the peptide. Further, a bioassay can be used to monitor the composition's translocation. For example, using a bioactive molecules such as erythropoietin, included in a composition, the increase in hemoglobin or hematocrit can be measured. Similarly, by using a bioactive molecule such as insulin coupled with a composition, the drop in blood glucose level can be measured.

“Effective translocation” as used herein means that introduction of the composition to a biological barrier results in at least 5%, but preferably at least 10%, and even more preferably at least 20%, translocation of the effector across the biological barrier. The at least one effector of the composition is selectively encapsulated in such a way that introduction of the composition to a biological barrier results in translocation of the encapsulated effector only, i.e., no other molecules concomitantly administered in a non-encapsulated or free form are translocated across the barrier.

As used herein, the term “encapsulation” refers to the introduction of the at least one effector to the hydrophobic composition. The method of encapsulation can involve complex formation of at least one effector with at least one amphipathic counter ion, and dissolution in water or in an at least partially water soluble solvent. The composition can be further supplemented by a protein stabilizer, a penetrating peptide, and one or more pharmaceutically acceptable hydrophobic agents. Any one or more of the components of the composition inay be lyophilized at various stages of the encapsulation process.

A hydrophobic agent can be a single molecule or a combination of hydrophobic molecules, like aliphatic, cyclic, or aromatic molecules. Examples of aliphatic hydrophobic agents include mineral oil, paraffin, fatty acids, mono-, di-, or tri-glycerides, ethers, or esters. Examples of tri-glycerides include long chain triglycerides, medium chain triglycerides, and short chain triglycerides. Specific examples of suitable triglycerides include tributyrin, trihexanoin, trioctanoin, and tricaprin (1,2,3-tridecanoyl glycerol). Examples.of cyclic hydrophobic agents include terpenoids, cholesterol, cholesterol derivatives and cholesterol esters of fatty acids. An example of an aromatic hydrophobic agent includes benzyl benzoate. At least partially water soluble solvents include, for example, n-butanol, isoamyl (=isopentyl) alcohol, DMF, DMSO, iso-butanol, iso-propanol, propanol, ethanol, ter-butanol, polyols, ethers, amides, esters, or various mixtures thereof.

As used herein, the term “effector” refers to any cationic or anionic impermeable molecule or compound of, for example, biological, therapeutic, pharmaceutical, or diagnostic tracing. An anionic impermeable molecule can consist of nucleic acids (ribonucleic acid, deoxyribonucleic acid) from various origins, and particularly of human, viral, animal, eukaryotic or prokaryotic, plant, synthetic origin, etc. A nucleic acid of interest may be of a variety of sizes, ranging from, for example, a simple trace nucleotide to a genome fragment, or an entire genome. It may be a viral genome or a plasmid.

Alternatively, the effector of interest can be a protein, such as, for example, an enzyme, a hormone, a cytokine, an apolipoprotein, a growth factor, a bioactive molecule, an antigen, or an antibody, etc. As used herein, the term “bioactive molecule” refers to those compounds that have an effect on or elicit a response from living cells or tissues. A non-limiting example of a bioactive molecule is a protein. Other examples of the bioactive molecule include, but are not limited to, insulin, erythropoietin (EPO), glucagon-like peptide 1 (GLP-1), αMSH, parathyroid hormone (PTH), growth hormone, calcitonin, interleukin-2 (IL-2), α1-antitrypsin, granulocyte/monocyte colony stimulating factor (GM-CSF), granulocyte colony stimulating factor (G-CSF), T20, anti-TNF antibodies, interferon α, interferon β, interferon γ, lutenizing hormone (LH), follicle-stimulating hormone (FSH), enkephalin, dalargin, kyotorphin, basic fibroblast growth factor (bFGF), hirudin, hirulog, lutenizing hormone releasing hormone (LHRH) analog, brain-derived natriuretic peptide (BNP), glatiramer acetate (Copolymer-1), or neurotrophic factors. The effector of interest can also be a glycosaminoglycan including, but not limited to, heparin, heparan sulfate, chondroitin sulfate, dermatan sulfate, and hyaluronic acid. The effector of interest can further be a nucleic acid such as DNA, RNA, a DNA mimetic, or an RNA mimetic. Additionally, the effector can be a pharmaceutically active agent, such as, for example, a toxin, a therapeutic agent, or an antipathogenic agent, such as an antibiotic, an antiviral, an antifungal, or an anti-parasitic agent. The effector of interest can itself be directly active or can be activated in situ by the peptide, by a distinct substance, or by environmental conditions.

The terms “pharnaceutically active agent” and “therapeutic agent” are used herein interchangeably to refer to a chemical material or compound, which, when administered to an organism, induces a detectable pharmacologic and/or physiologic effect.

The hydrophobic compositions according to the present invention are characterized by the fact that their penetration capacity is virtually independent of the nature of the effector that is included within it.

“Counter ions” according to this invention can include, for example, anionic or cationic amphipathic molecules, i.e., those having both polar and nonpolar domains, or both hydrophilic and hydrophobic properties. Anionic or cationic counter ions of this invention are ions that are negatively (anionic) or positively (cationic) charged and can include a hydrophobic moiety. Under appropriate conditions, anionic or cationic counter ions can establish electrostatic interactions with cationic or anionic impermeable molecules, respectively. The formation of such a complex can cause charge neutralization, thereby creating a new uncharged entity, with further hydrophobic properties in the case of an inherent hydrophobicity of the counter ion.

Suitable anionic counter ions include ions with negatively charged residues such as carboxylate, sulfonate or phosphonate anions, and can further contain a hydrophobic moiety. Examples of such anionic counter ions include sodium dodecyl sulphate, dioctyl sulfosuccinate and other anionic compounds derived from organic acids.

Suitable cationic counter ions include quaternary amine derivatives, such as benzalkonium derivatives or other quaternary amines, which can be substituted by hydrophobic residues. In general, quaternary amines contemplated by the invention have the structure: 1-R1-2-R2-3-R3-4-R4-N, wherein R1,2,3, or 4 are alkyl or aryl derivatives. Further, quaternary amines can also be ionic liquid forming cations, such as imidazolium derivatives, pyridinium derivatives, phosphonium compounds or tetralkylammonium compounds.

Ionic liquids are salts composed of cations such as imidazolium ions, pyridinium ions and anions such as BF₄⁻; PF₆⁻ and are liquid at relatively low temperatures. Ionic liquids are characteristically in liquid state over extended temperature ranges, and have high ionic conductivity. Other favorable characteristic properties of the ionic liquids include non-flammability, high thermal stability, relatively low viscosity, and essentially no vapor pressure. When an ionic liquid is used as a reaction solvent, the solute is solvated by ions only, thus creating a totally different environment from that when water or ordinary organic solvents are used. This enables high selectivity, applications of which are steadily expanding. Some examples are in the Friedel-Crafts reaction, Diels-Alder reaction, metal catalyzed asymmetric synthesis and others. Furthermore, some ionic liquids have low solubility in water and low polar organic solvents, enabling their recovery after reaction product is extracted with organic solvents. Ionic liquids are also used electrochemically, due to their high ion-conductivity, for example as electrolytes of rechargeable batteries.

As mentioned above, in one preferred embodiment, the counter ion can be an ionic liquid forming cation. For example, imidazolium derivatives have the general structure of 1-R1-3-R2-imidazolium where R1 and R2 can be linear or branched alkyls with 1 to 12 carbons. Such imidazolium derivatives can be further substituted for example by halogens or an alkyl group. Specific imidazolium derivatives include, but are not limited to, 1-ethyl-3-methylimidazolium, 1-butyl-3-methylimidazolium, 1-hexyl-3-methylimidazolium, 1-methyl-3-octylimidazolium, 1-methyl-3-(3,3,4,4,5,5,6,6,7,7,8,8,8-tridecafluoroctyl)-imidazolium, 1,3-dimethylimidazolium, and 1,2-dimethyl-3-propylimidazolium.

Pyridinium derivatives have the general structure of 1-R1-3-R2-pyridinium where R1 is a linear or branched alkyl with 1 to 12 carbons, and R2 is H or a linear or branched alkyl with 1 to 12 carbons. Such pyridinium derivatives can be further substituted for example by halogens or an alkyl group. Pyridinium derivatives include, but are not limited to, 3-methyl-1-propylpyridinium, 1-butyl-3-methylpyridinium, and 1-butyl-4-methylpyridinium.

The present invention relates to the use of the cationic component of ionic liquids. Unlike other ionic liquids, the salts of the cations according to the present invention are typically water soluble. For example, an anionic counterpart of the ionic liquid forming cation can be a halogen, such as chloride or bromide.

The compositions of this invention may further contain a stabilizer of protein structure. As descnbed above, stabilizers of protein structure are compounds that stabilize protein structure under aqueous or non-aqueous conditions. Stabilizers of protein structure can be polyanionic molecules, such as phytic acid and sucrose octasulfate, or polycationic molecules, such as spermine. Stabilizers of protein structure can also be uncharged polymers, such as polyvinylpyrrolidone and polyvinyl alcohol.

Phytic acid and its derivatives are biologically active compounds known to bind several proteins with high affinity. Phytic acid contains six phosphate residues attached to a cyclohexane ring, enabling it to bind several guanidinium groups of arginines. See for example Filikov et al., J. Comput. Aided Mol. Des. 12:229-240 (1998).

Any of the hydrophobic compositions of the invention may further contain a penetrating peptide. The use of small peptide carriers in the compositions described herein allow for high quality and purity and the potential for highly efficient delivery through biological barriers in an organism. The present invention employs a short peptide motif to create hydrophobic compositions to specifically transport macromolecules across biological barriers sealed by tight junctions.

In some embodiments, the present invention provides a peptide penetration system, i.e., a penetration composition, that specifically targets various tissues, especially epithelial and endothelial ones, for the delivery of drugs and other therapeutic agents across a biological barrier The peptide penetration system of the present invention uses conserved peptide sequences from various proteins involved in paracytosis to create a penetration composition capable of crossing biological barriers. For example, a peptide encoded by or derived from ORF HI0638 of Haemophilus influenzae facilitates penetration of this bacterium between human lung epithelial cells without compromising the epithelial barrier. The peptide sequence encoded by ORF HI0638 is conserved in common pathogenic bacteria or symbiotic bacteria including, for example, Haemophilus influenzae, Pasteurella multocida, Escherichia coli, Vibrio cholerae, Buchnera aphidicola, Pseudonmonas aeruginosa, and Xylella fastidiosa. A peptide homologous to the N-terminal sequence of HI0638 is also found in other bacteria including, for example, Rhizobium loti, Chlamydia pneumoniae, NprB from Bacillus subtilis, and pilins from Kingella dentrificains and Eikenella corrodens.

Furthermore, a similar peptide sequence is also conserved in proteins of eukaryotic origin such as the neurokinin receptor family proteins, including the human NK-1 and NK-2 receptors. It is known that the neurokinin receptor family is involved in the control of intercellular permeability including plasma extravasation and oedema formation. Extravasation, the leakage and spread of blood or fluid from vessels into the surrounding tissues, often follows inflammatory processes involved in tissue injury, allergy, burns and inflammation. In particular, when NK-1 receptors on blood vessels are activated, skin inflammation occurs due to an increase in vascular permeability. See Inoue, et al., Inflamm. Res., 45:316-323 (1996). The neurokinin NK-1 receptor also mediates dural and extracranial plasma protein extravasation, thereby implicating the NK-1 receptor in the pathophysiology of migraine headache. See O'Shaughnessy and Connor, Euro. J. of Pharm., 236:319-321 (1993).

The sequences of example penetrating peptides according to the invention are shown in Tables A and B.

TABLE A

| | | |
|---|---|---|
| Peptide/Organism | Sequence | SEQ ID NO |

| | | |
|---|---|---|
| Peptide 1: from ORF HI0638 | NYHDIVLALAGVCQSAKLVHQLA | (SEQ ID NO:1) |
| Haemophilus influenzae | | |
| Peptide 2: from PM1850 | NYYDITLALAGVCQAAKLVQQFA | (SEQ ID NO:2) |
| Pasteurella mnultocida | | |
| Peptide 3: from YCFC | NYYDITLALAGICQSARLVQQLA | (SEQ ID NO:3) |
| Excherichia coli | | |
| Peptide 4: from VC1127 | AIYDRTIAFAGICQAVALVQQVA | (SEQ ID NO:4) |
| Vibrio cholerae | | |
| Peptide 5: from BU262 | KIHLITLSLAGICQSAHLVQQLA | (SEQ ID NO:5) |
| Buchnera aphidicola | | |
| Peptide 6: from PA2627 | DPRQQLIALGAVFESAALVDKLA | (SEQ ID NO:6) |
| Pseudomonas aeruginosa | | |
| Peptide 7: from XF1439 | LIDNRVLALAGVVQALQQVRQIA | (SEQ ID NO:7) |
| Xylella fastidiosa | | |
| Peptide 8: from MLR0187 | NLPPIVLAVIGICAAVFLLQQYV | (SEQ ID NO:8) |
| Rhizobium loti | | |
| Peptide 9: from Human NK-2 | NYFIVNLALADLCMAAFNAAFNF | (SEQ ID NO:9) |
| Receptor | | |
| Peptide 10: from CPN0710/C | TAFDFNKMLDGVCTYVKGVQQYL | (SEQ ID NO:10) |
| Chlamydia pneumoniae | | |
| Peptide 11: from MLR4119 | RAILIPLALAGLCQVARAGDISS | (SEQ ID NO:11) |
| Rhizobium loti | | |
| Peptide 12: from NprB | MRNLTKTSLLLAGLCTAAQMYFVTH | (SEQ ID NO:12) |
| Bacillus subtilis | | |
| Peptide 13: from Pilin | IELMIVIAIIGILAAIALPAYQEYV | (SEQ ID NO:13) |
| Kingella dentrificans | | |
| Peptide 14: from Pilin | IELMIVIAIIGILAAIALPAYQDYV | (SEQ ID NO:14) |
| Eikenella corrodens | | |
| Peptide 15: from zonula | ASFGFCIGRLCVQDGF | (SEQ ID NO:15) |
| occludens toxin (ZOT) | | |
| Peptide 29: from Human NK-1 | NYFLVNLAFAEASMAAFNTVVNF | (SEQ ID NO:24) |
| Receptor | | |
| Peptide 30: from YCFC | MNYYDITLALAGICQSARLVQQLA | (SEQ ID NO:25) |
| Escherichia coli | | |
| Peptide 31: from YCFC | MYYDITLALAGICQSARLVQQLA | (SEQ ID NO:26) |
| Escherichia coli | | |
| Peptide 32: from YCFC | MYDITLALAGICQSARLVQQLA | (SEQ ID NO:27) |
| Escherichia coli | | |
| Peptide 33: from NprB | MRNLTRTSLLLAGLCTAAQMVFV | (SEQ ID NO:28) |
| Bacillus subtilis | | |
| Peptide 34: from ORF HI0638 | NYHDIVLALAGVCQSARLVHQLA | (SEQ ID NO:29) |
| Haemohilus influenzae | | |

TABLE B

| | SEQ | |
|---|---|---|
| Peptide's | ID | |
| name | NO. | Sequence |
| IBW-002 | 22 | AcNYYDITLALAGICQSARLVQQLAGGGKGGKNH₂ |
| IBW-003 | 30 | AcNLPPIVLAVIGICAAVFLLQQYVGGGKGGKNH₂ |
| IBW-004 | 31 | AcNYFIVNLALADLCMAAFNAAFNFGGGKGGKNH₂ |
| IBW-005 | 32 | AcMRNLTRTSLLLAGLCTAAQMVFVGGGKGGKNH₂ |
| IBW-006 | 33 | AcNYHDIVLALAGVCQSARLVHQLAGGKGGKNH₂ |
| IBW-007 | 34 | AcNYFLVNLAFAEASMAAFNTVVNFGGKGGKNH₂ |
| IBW-002V1 | 35 | AcMNYYDITLALAGICQSARLVQQLAGGGKGGKNH₂ |
| IBW-002V2 | 36 | AcMYYDITLALAGICQSARLVQQLAGGGKGGKNH₂ |
| IBW-002V3 | 37 | AcMYDITLALAGICQSARLVQQLAGGGKGGKNH₂ |

The penetrating peptides of the instant invention also include peptides containing at least 12 contiguous amino acids of any of the peptides defined by SEQ ID NOS:1-15 and 24-29.

The peptides described herein serve as the basis for the design of therapeutic “cargos”, namely the coupling of the carriers (“penetrating peptide”) with one or more therapeutic agents (“effectors”). Preferably a non-covalent bond is used to couple a penetrating peptide to one or more effectors. The penetrating peptide can be attached to a linker to which imaging compounds can be covalently attached, for example through free amino groups of lysine residues. Such a linker may include, but is not limited to, the amino acid sequence GGKGGK (SEQ ID NO:16), alternatively referred to herein as IBW-001).

Compositions of this invention that include a penetrating peptide involve the coupling of the penetrating peptide to the effector, either directly or indirectly. As used herein, the term “coupled” is meant to include all such specific interactions that result in two or more molecules showing a preference for one another relative to some third molecule, including any type of interaction enabling a physical association between an effector and a penetrating peptide. Preferably, this includes, but is not limited to, electrostatic interactions, hydrophobic interactions and hydrogen bonding, but does not include non-specific associations such as solvent preferences. The association must be sufficiently strong so that the effector does not dissociate before or during penetration of the biological barrier.

Furthermore, the coupling of the effector to the penetrating peptide can be achieved indirectly via a mediator. For example, such a mediator can be a large hydrophobic molecule, such as, for example, free fatty acids, mono-, di-, or tri-glycerides, ethers, or cholesterol esters of fatty acids, that binds the effector-counter ion complex, on the one hand, and the hydrophobized penetrating peptide, on the other hand.

Also included in the invention are methods of producing the compositions described herein. For example, the effector and the counter ion can be lyophilized or freeze dried together and then reconstituted under preferred solvent surroundings. Any one or more of the protein stabilizers, the penetrating peptides, and/or any other constituent of the pharmaceutical excipient or carrier can be optionally added with the effector and counter ion during the lyophilization. Other components of the composition can also be optionally added during reconstitution of the lyophilized materials. Such optional components can include, for example, pluronic F-68, Aprotinin, Solutol HS-15, N-Acetyl Cysteine, and/or Tricaprin.

For example, a penetrating peptide or effector of the composition can be produced by standard recombinant DNA techniques known in the art.

As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

Recombinant expression vectors comprise a nucleic acid in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The term “regulatory sequence” is intended to include promoters, enhancers and other expression control elements (e.g., polyadenylation signals). Such regulatory sequences are described, for example, in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. Expression vectors can be introduced into host cells to thereby produce proteins or peptides encoded by nucleic acids as described herein (e.g., penetrating peptides).

Recombinant expression vectors can be designed for expression of penetrating peptides or effectors of the invention in prokaryotic or eukaryotic cells. For example, penetrating peptides or effectors can be expressed in bacterial cells such as Escherichia coli, insect cells (using baculovirus expression vectors), yeast cells or mammalian cells. Suitable host cells are discussed further in Goeddel, GENE EXPRESSION TECHNOLOGY: METHODS IN ENZYMOLOGY 185, Academic Press, San Diego, Calif (1990). Alternatively, the recombinant expression vector can be transcribed and translated in vitro, for example using T7 promoter regulatory sequences and T7 polymerase.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification.

Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

One strategy to maximize recombinant protein expression in E. coli is to express the protein in a host bacteria with an impaired capacity to proteolytically cleave the recombinant protein. See, e.g., Gottesman, GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (see, e.g., Wada, et al., 1992. Nucl. Acids Res. 20: 2111-2118). Such alteration of nucleic acid sequences encoding the penetrating peptides or compositions of the invention can be carried out by standard DNA synthesis techniques.

In another embodiment, the expression vector is a yeast expression vector. Examples of vectors for expression in yeast Saccharoinyces cerivisae include pYepSec1(Baldari, et al., 1987. EMBO J. 6: 229-234), pMFa (Kurjan and Herskowitz, 1982. Cell 30: 933-943), pJRY88 (Schultz et al., 1987. Gene 54: 113-123), pYES2 (Invitrogen Corporation, San Diego, Calif.), and picZ (Invitrogen Corp, San Diego, Calif).

Alternatively, a penetrating peptide or effectors of the invention can be expressed in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In yet another embodiment, a nucleic acid encoding the penetrating peptides and effectors of the invention are expressed in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells see, e.g. Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Banerji, et al., 1983. Cell 33: 729-740; Queen and Baltimore, 1983. Cell 33: 741-748), neuron-specific promoters (e.g., the neurofilament promoter, Byme and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546).

The invention further provides a recombinant expression vector comprising a DNA molecule encoding the penetrating peptides and effectors of the invention cloned into the expression vector in an antisense orientation. That is, the DNA molecule is operatively-linked to a regulatory sequence in a manner that allows for expression (by transcription of the DNA molecule) of an RNA molecule that is antisense to the penetrating peptide mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see, e.g., Weintraub, et al., “Antisense RNA as a molecular tool for genetic analysis,” Reviews-Trends in Genetics, Vol. 1(1) 1986.

Another aspect of the invention pertains to host cells into which a recombinant expression vector has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, the penetrating peptide or effectors can be expressed in bacterial cells such as E. coli, insect cells, yeast or mammalian cells (such as Chinese hamster ovary cells (CHO) or COS cells). Other suitable host cells are known to those skilled in the art

Vector DNA can be introduced into prokaryotic or eukaryotic cells via conventional transformation or transfection techniques. As used herein, the terms “transformation” and “transfection” are intended to refer to a variety of art-recognized, techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest Various selectable markers include those that confer resistance to drugs, such as G418, hygromycin and methotrexate. Nucleic acid encoding a selectable marker can be introduced into a host cell on the same vector as that encoding the penetrating peptide or composition, or can be introduced on a separate vector. Cells stably transfected with the introduced nucleic acid can be identified by drug selection (e.g., cells that have incorporated the selectable marker gene will survive, while the other cells die).

A host cell, such as a prokaryotic or eukaryotic host cell in culture, can be used to produce (i.e., express) a penetrating peptide or an effector of the invention. Accordingly, the invention further provides methods for producing penetrating peptides or effectors using the host cells. In one embodiment, the method comprises culturing the host cell (into which a recombinant expression vector encoding a penetrating peptide or an effector has been introduced) in a suitable medium such that the penetrating peptide or effector is produced. In another embodiment, the method further comprises isolating the penetrating peptide or composition from the medium or the host cell.

The penetrating peptides and effectors of the invention can also be produced using solid-phase peptide synthesis methods known in the art. For example, a penetrating peptide can be synthesized using the Merrifield solid-phase synthesis method. (See e.g., Merrifield, R. B., J. Am. Chem. Soc. 85:2149 (1963); ENCYCLOPEDIA OF MOLECULAR BIOLOGY 806 (1st ed. 1994)). In this method, the C-terminal amino acid is attached to an insoluble polymeric support resin (e.g., polystyrene beads), thereby forming an immobilized amino acid. To avoid unwanted reactions as the C-terminal amino acid is attached to the resin, the amino group of the C-terminal amino acid is protected or “blocked” using, for example, a tert-butyloxylcarbonyl (t-BOC) group. The blocking group, e.g., t-BOC, on the immobilized amino acid is then removed by adding a dilute acid to the solution. Before a second amino acid is attached to the immobilized peptide chain, the amino-group of the second amino acid is blocked, as described above, and the α-carboxyl group of the second amino acid is activated through a reaction with dicyclohxylcarbdiimide (DCC). The activated α-carboxyl group of the second amino acid then reacts with the free amino group of the immobilized amino acid to form a peptide bond. Additional amino acids are then individually added to the terminal amino acid of the immobilized peptide chain according to the required sequence for the desired penetrating peptide or composition. Once the amino acids have been added in the required sequence, the completed peptide is released from the resin, such as for example, by using hydrogen fluoride, which does not attack the peptide bonds.

The penetrating peptides or effectors of the invention can also be synthesized using Fmoc solid-phase peptide synthesis. (See e.g., University of Illinois at Urbana-Champaign Protein Sciences Facility, Solid-Phase Peptide Syntlesis (SPPS), at http://www.biotech.uiuc.edu/spps.htm). In this method, the C-terminal amino acid is attached to an insoluble polymeric support resin (e.g., polystyrene beads, cross-linked polystyrene resins, etc.), such as for example, via an acid labile bond with a linker molecule. To avoid unwanted reactions as the C-terminal amino acid is being attached to the resin, the amino group of the C-terminal amino acid is blocked using an Fmoc group. The blocking group, e.g., Fmoc, on the terminal amino acid of the immobilized amino acid is then removed by adding a base to the solution. Side chain functional groups are also protected using any base-stable, acid-labile groups to avoid unwanted reactions. Before the second amino acid is attached to the immobilized amino acid, the amino-group of the second amino acid is blocked, as described above, and the α-carboxyl group of each successive amino acid is activated by creating an N-hydrobenzotriazole (HOBt) ester in situ. The activated α-carboxyl group of the second amino acid and the free amino group of the immobilized amino acid then react, in the presence of a base, to form a new peptide bond. Additional amino acids are then successively added to the terminal amino acid of the immobilized peptide chain, until the desired peptide has been assembled. Once the necessary amino acids have been attached, the peptide chain can be cleaved from the resin, such as for example, by using a mixture of trifluoroacetic acid (TFA) and scavengers (e.g., phenol, thioanisol, water, ethanedithiol (EDT) and triisopropylsilan (TIS)) that are effective to neutralize any cations formed as the protecting groups attached to the side chain functional groups of the assembled peptide chain are removed.

It is well known to those skilled in the art that proteins can be further chemically modified to enhance the protein half-life in circulation. By way of non-limiting example, polyethylene glycol (PEG) residues can be attached to the penetrating peptides or effectors of the invention. Conjugating biomolecules with PEG, a process known as pegylation, is an established method for increasing the circulating half-life of proteins. Polyethylene glycols are nontoxic water-soluble polymers that, because of their large hydrodynamic volume, create a shield around the pegylated molecule, thereby protecting it from renal clearance, enzymatic degradation, as well as recognition by cells of the immune system.

Agent-specific pegylation methods have been used in recent years to produce pegylated molecules (e.g., drugs, proteins, agents, enzymes, etc.) that have biological activity that is the same as, or greater than, that of the “parent” molecule. These agents have distinct in vivo pharmacokinetic and pharmacodynamic properties, as exemplified by the self-regulated clearance of pegfilgrastim, the prolonged absorption half-life of pegylated interferon alpha-2a. Pegylated molecules have dosing schedules that are more convenient and more acceptable to patients, which can have a beneficial effect on the quality of life of patients. (See e.g., Yowell S. L. et al., Cancer Treat Rev 28 Suppl. A:3-6 (April 2002)).

The invention also includes methods of contacting biological barrier with compositions of the invention in an amount sufficient to enable efficient penetration of the compositions through the barrier. The compositions of this invention can be provided in vitro, ex vtvo, or in vivo. Furthermore, the composition according to this invention may be capable of potentializing the biological activity of the coupled substance. Therefore, these compositions can be used to increase the biological activity of the effector.

In addition to the hydrophobic composition, the invention also provides a pharmaceutically acceptable base or acid addition salt, hydrate, ester, solvate, prodrug, metabolite, stereoisomer, or mixture thereof. The invention also includes pharmaceutical formulations comprising a hydrophobic composition in association with a pharmaceutically acceptable carrier, diluent, protease inhibitor, surface active agent, or excipient. A surface active agent can include, for example, poloxamers, Solutol HS15, cremophore, or bile acids/salts.

Salts encompassed within the term “pharmaceutically acceptable salts” refer to non-toxic salts of the compounds of this invention which are generally prepared by reacting the free base with a suitable organic or inorganic acid or solvent to produce “pharmaceutically-acceptable acid addition salts” of the compounds described herein. These compounds retain the biological effectiveness and properties of the free bases. Representative of such salts are the water-soluble and water-insoluble salts, such as the acetate, amsonate (4,4-diaminostilbene-2, 2′-disulfonate), benzenesulfonate, benzoate, bicarbonate, bisulfate, bitartrate, borate, bromide, butyrate, calcium edetate, camsylate, carbonate, chloride, citrate, clavulariate, dihydrochloride, edetate, edisylate, estolate, esylate, fumarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methylene-bis-2-hydroxy-3-naphthoate, embonate), pantothenate, phosphate/diphosphate, picrate, polygalacturonate, propionate, p-toluenesulfonate, salicylate, stearate, subacetate, succinate, sulfate, sulfosaliculate, suramate, tannate, tarrate, teoclate, tosylate, triethiodide, and valerate salts.

According to the methods of the invention, a patient, i.e, a human patient, can be treated with a pharmacologically or therapeutically effective amount of a hydrophobic composition. The term “pharmacologically or therapeutically effective amount” means that amount of a drug or pharmaceutical agent (the effector) that will elicit the biological or medical response of a tissue, system, animal or human that is being sought by a researcher or clinician.

The invention also includes pharmaceutical compositions suitable for introducing an effector of interest across a biological barrier. The compositions are preferably suitable for internal use and include an effective amount of a pharmacologically active compound of the invention, alone or in combination, with one or more pharmaceutically acceptable carriers. The compounds are especially useful in that they have very low, if any, toxicity.

Preferred pharmaceutical compositions are tablets and gelatin capsules, enteric-coated, comprising the active ingredient together with a) diluents, e.g. lactose, dextrose, sucrose, mannitol, sorbitol, cellulose and/or glycine; b) protease inhibitors including, but not limited to, aprotinin, Bowman-Birk inhibitor, soybean trypsin inhibitor, chicken ovomucoid, chicken ovoinhibitor, human pancreatic trypsin inhibitor, camostate mesilate, flavonoid inhibitors, antipain, leupeptin,p-aminobenzamidine, AEBSF, TLCK, APMSF, DFP, PMSF, poly(acrylate) derivatives, chymostatin, benzyloxycarbonyl-Pro-Phe-CHO; FK-448, sugar biphenylboronic acids complexes, β-phenylpropionate, elastatinal, methoxysuccinyl-Ala-Ala-Pro-Val-chloromethylketone (MPCMK), EDTA, chitosan-EDTA conjugates, amino acids, di-peptides, tripeptides, amastatin, bestatin, puromycin, bacitracin, phosphinic acid dipeptide analogues, α-aminoboronic acid derivatives, Na-glycocholate, 1,10-phenantroline, acivicin, L-serine-borate, thiorphan, and phosphoramidon ; c) lubricants, e.g., silica, talcum, stearic acid, its magnesium or calcium salt, poloxamer and/or polyethyleneglycol; for tablets also d) binders, e.g., magnesium aluminum silicate, starch paste, gelatin, tragacanth, methylcellulose, sodium carboxymethylcellulose and/or polyvinylpyrrolidone; if desired e) disintegrants, e.g., starches, agar, alginic acid or its sodium salt, or effervescent mixtures; and/or f) absorbents, colorants, flavors and sweeteners. The compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. The compositions are prepared according to conventional mixing, granulating or coating methods, respectively, and contain about 0.001 to 75%, preferably about 0.01 to 10%, of the active ingredient

Administration of the active compounds and salts described herein can be via any of the accepted modes of administration for therapeutic agents. These methods include oral, buccal, anal, rectal, bronchial, nasal, sublingual, parenteral, transdermal, pulmonary, or topical administration modes. As used herein, the term “parenteral” refers to injections given through some other route than the alimentary canal, such as subcutaneously, intramuscularly, intraorbitally (i.e., into the eye socket or behind the eyeball), intracapsularly, intraspirally, intrasternally or intraveneously.

Depending on the intended mode of administration, the compositions may be in solid, semi-solid or liquid dosage form, such as, for example, tablets, suppositories, pills, time-release capsules, powders, liquids, suspensions, aerosol or the like, preferably in unit dosages. The compositions will include an effective amount of active compound or the pharmaceutically acceptable salt thereof, and in addition, may also include any conventional pharmaceutical excipients and other medicinal or pharmaceutical drugs or agents, carriers, adjuvants, diluents, protease inhibitors, etc., as are customarily used in the pharmaceutical sciences.

For solid compositions, excipients include pharmaceutical grades of mannitol, lactose, starch, magnesium stearate, sodium saccharin, talcum, cellulose, glucose, sucrose, magnesium carbonate, and the like may be used. The active compound defined above, may be also formulated as suppositories using for example, polyalkylene glycols, for example, propylene glycol, as the carrier.

Liquid compositions can, for example, be prepared by dissolving, dispersing, etc. The active compound is dissolved in or mixed with a pharmaceutically pure solvent such as, for example, water, saline, aqueous dextrose, glycerol, ethanol, and the like, to thereby form the solution or suspension.

If desired, the pharmaceutical composition to be administered may also contain minor amounts of non-toxic auxiliary substances such as wetting or emulsifying agents, pH buffering agents, and other substances such as for example, sodium acetate, triethanolamine oleate, etc.

Those skilled in the art will also recognize that the hydrophobic compositions of the instant invention can also be used as a mucosal, ie. oral, nasal, rectal, vaginal, or bronchial, vaccine having an antigen, to which vaccination is desired, serve as the effector. Such a vaccine may include a composition including a desired antigenic sequence, including, but not limited to, the protective antigen (PA) component of Anthrax or the Hepatitis B surface antigen (HBs) of Hepatitis B. This composition is then orally or nasally administered to a subject in need of vaccination.

An “antigen” is a molecule or a portion of a molecule capable of stimulating an immune response, which is additionally capable of inducing an animal or human to produce antibody capable of binding to an epitope of that antigen. An “epitope” is that portion of any molecule capable of being recognized by and bound by a major histocompatability complex (“MHC”) molecule and recognized by a T cell or bound by an antibody. A typical antigen can have one or more than one epitope. The specific recognition indicates that the antigen will react, in a highly selective manner, with its corresponding MHC and T cell, or antibody and not with the multitude of other antibodies which can be evoked by other antigens.

A peptide is “immunologically reactive” with a T cell or antibody when it binds to an MHC and is recognized by a T cell or binds to an antibody due to recognition (or the precise fit) of a specific epitope contained within the peptide. Immunological reactivity can be determined by measuring T cell response in vitro or by antibody binding, more particularly by the kinetics of antibody binding, or by competition in binding using known peptides containing an epitope against which the antibody or T cell response is directed as competitors.

Techniques used to determine whether a peptide is immunologically reactive with a T cell or with an antibody are known in the art. Peptides can be screened for efficacy by in vitro and in vivo assays. Such assays employ immunization of an animal, e.g., a mouse, a rabbit or a primate, with the peptide, and evaluation of the resulting antibody titers.

Also included within the invention are vaccines that can elicit the production of secretory antibodies (IgA) against the corresponding antigen, as such antibodies serve as the first line of defense against a variety of pathogens. Oral or nasal i.e., mucosal, vaccination, which have the advantage of being non-invasive routes of administration, are the preferred means of immunization for obtaining secretory antibodies, although those skilled in the art will recognize that the vaccination can be administered in a variety of ways, e.g., orally, topically, or parenterally, i.e., subcutaneously, intraperitoneally, by viral infection, intravascularly, etc.

The compositions of the present invention can be administered in oral dosage forms such as tablets, capsules (each including timed release and sustained release formulations), pills, powders, granules, elixirs, tinctures, suspensions, syrups and emulsions.

The dosage regimen utilizing the compounds is selected in accordance with a variety of factors including type, species, age, weight, sex and medical condition of the patient, the severity of the condition to be treated; the route of administration; the renal and hepatic function of the patient; and the particular compound or salt thereof employed. An ordinarily skilled physician or veterinarian can readily determine and prescribe the effective amount of the drug required to prevent, counter or arrest the progress of the condition.

Oral dosages of the present invention, when used for the indicated effects, may be provided in the form of scored tablets containing 0.005, 0.01, 0.025, 0.05, 0.1, 0.25, 0.5, 1.0, 2.5, 5.0, 10.0, 15.0, 25.0, 50.0, 100.0, 250.0, 500.0 or 1000.0 mg of active ingredient.

Compounds of the present invention may be administered in a single daily dose, or the total daily dosage may be administered in divided doses of two, three or four times daily. Furthermore, preferred compounds for the present invention can be administered in bucal form via topical use of suitable bucal vehicles, bronchial form via suitable aerosols or inhalants, intranasal form via topical use of suitable intranasal vehicles, or via transdermal routes, using those forms of transdermal skin patches well known to those of ordinary skill in that art. To be administered in the form of a transdermal delivery system, the dosage administration will, of course, be continuous rather than intermittent throughout the dosage regimen. Other preferred topical preparations include creams, ointments, lotions, aerosol sprays and gels, wherein the concentration of active ingredient would range from 0.1% to 15%, w/w or w/v.

The compounds herein described in detail can form the active ingredient, and are typically administered in admixture with suitable pharmaceutical diluents, excipients or carriers (collectively referred to herein as “carrier” materials) suitably selected with respect to the intended form of administration, that is, oral tablets, capsules, elixirs, syrups and the like, and consistent with conventional pharmaceutical practices.

For instance, for oral administration in the form of a tablet or capsule, the active drug component can be combined with an oral, non-toxic pharmaceutically acceptable inert carrier such as ethanol, glycerol, water and the like. Moreover, when desired or necessary, suitable binders, lubricants, protease inhibitors, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, poloxamer, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methylcellulose, agar, bentonite, xanthan gum and the like.

The compounds of the present invention may also be coupled with soluble polymers as targetable drug carriers. Such polymers can include polyvinylpyrrolidone, pyran copolymer, polyhydroxypropyl-methacrylamide-phenol, polyhydroxyethylaspanamidephenol, or polyethyleneoxidepolylysine substituted with palmitoyl residues. Furthermore, the compounds of the present invention may be coupled to a class of biodegradable polymers useful in achieving controlled release of a drug, for example, polylactic acid, polyepsilon caprolactone, polyhydroxy butyric acid, polyorthoesters, polyacetals, polydihydropyrans, polycyanoacrylates and cross-linked or amphipathic block copolymers of hydrogels.

Any of the above pharmaceutical compositions may contain 0.01-99%, preferably 0.1-10% of the active compounds as active ingredients.

The following EXAMPLES are presented in order to more fully illustrate the preferred embodiments of the invention. These EXAMPLES should in no way be construed as limiting the scope of the invention, as defined by the appended claims.

### EXAMPLES

### Example 1

### Utilization of Selective Encapsulation to Enable the Effective Translocation of Insulin Across an Epithelial Barrier

a) Composition for Translocation of Insulin Using BKC as the Counter Ion:

The composition was prepared by lyophilizing bovine insulin, the counter ion benzalkonium chloride (BKC), and phytic acid in a concentration ration of 1:0.5:0.5, and then reconstituting them with 0.5% tricaprin in ethanol, and adding a benzyl benzoate: butanol mixture in a ration of 1:11. Additional components of the composition are specified in Table 1.

TABLE 1
Composition for insulin translocation

| Insulin | 1 | mg/ml |
|---|---|---|
| Benzalkonium Chloride (BKC) | 0.5 | mg/ml |
| Phytic Acid | 0.5 | mg/ml |
| Tricaprine | 0.5 | mg/ml |
| Benzyl Benzoate:Butanol 1:11 | 60 | μl/ml |
| Pluronic F-68 | 2% | |
| Aprotinin | 100 | μl/ml |
| Solutol HS-15 (SHS) | 2% | |
| N-Acetyl Cysteine (NAC) | 50 | μg |
| Acetate Buffer | 20 | mM |
| Arginine | 20 | mg/ml |

Five male SD rats, 175-200 gr, were deprived of food, 18 hours prior to the experiment The animals were divided into 2 groups, and anesthetized by a solution of 85% ketamine, 15% xylazine, 0.1 ml/100 g of body weight. Each preparation was administered either i.p. (200 ul/rat, containing 1.14 IU insulin) or rectally (200 ul/mouse, containing 5.7 IU insulin). Blood glucose levels were measured at various time intervals post administration, in blood samples drawn from the tip of the tail. (See

TABLE 2

| | | Glucose, mg/dl, measured at follow times | | | | | |
|---|---|---|---|---|---|---|---|
| | | after injection | | | | | |
| | Route of | | 15 | 30 | 45 | 60 | 90 |
| Rat # | administration | 0 | min | min | min | min | min |
| 1 | 200 μl i.p. | 76 | 75 | 23 | <20 | <20 | 21 |
| | 1.14 U/rat | | | | | | |
| 2 | 200 μl i.p. | 108 | 108 | 92 | 68 | 77 | 75 |
| | 1.14 U/rat | | | | | | |
| 3 | 200 μl rectal | 91 | 127 | 24 | <20 | <20 | 30 |
| | 5.7 U/rat | | | | | | |
| 4 | 200 μl rectal | 81 | 61 | 27 | <20 | 22 | 25 |
| | 5.7 U/rat | | | | | | |
| 5 | 200 μl rectal | 87 | 64 | 30 | 21 | <20 | <20 |
| | 5.7 U/rat | | | | | | |

As can be seen above, after the composition was administered rectally, glucose levels dropped gradually and significantly, indicating insulin absorption from the intestine into the blood stream.

b) Composition for Translocation of Insulin Using BKC as the Counter Ion and a Penetrating Peptide:

SEQ ID NO: 36 (also called IBW-002V2) was hydrophobized via acylation of the free amino groups of the two lysine residues at the C-terminus of the penetrating peptide with a myristoyl. Acylation with myristoyl was achieved by incubating the peptide with myristoyl chloride in a molar ratio of 1:10, under basic pH conditions in the presence of appropriate solvents (benzyl benzoate and di-methyl formamide, with 1% bicarbonate). The hydrophobized peptide was then incorporated into the 15 composition, which further contained a lyophilizate of (1) insulin, (2) the counter cation Benzalkonium Chloride (BKC), and (3) phytic acid at a ratio of 1:0.5:0.5. Additional components of the composition are specified in Table 3.

TABLE 3
A composition for insulin translocation

Hydrophobized Peptide
Human Insulin
Benzalkonium Chloride (BKC)
Phytic Acid
NaOH
Acetic acid
Sodium Acetate
L-arginine
Pluronic F-68
Aprotinin
Solutol HS-15 (SHS)
N-Acetyl Cysteine (NAC)
Tricaprine
Ethanol

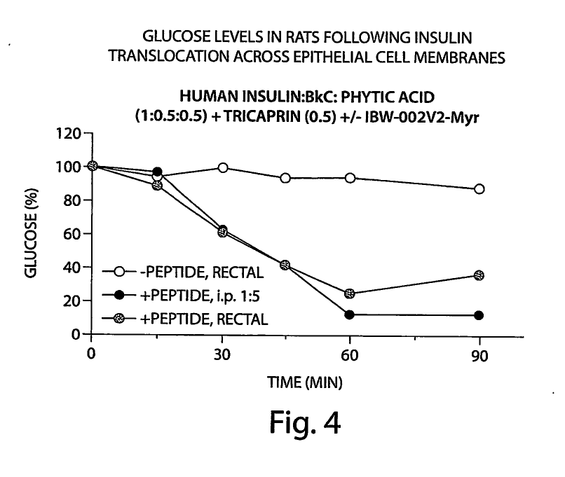

Twelve male SD rats, 160-190 gr, were deprived of food, 18 hours prior to the experiment. The animals were divided into groups. The preparations were administered as follows: Rats #1,2—rectal PBS 200 ul, rats #3,4—rectal 200 ul composition as specified above without peptide (5 IU insulin), rat #5—i.p. 200 ul composition with peptide (1 IU insulin), rats #6,7—rectal 200 ul composition with peptide (5 IU insulin). Blood glucose levels were measured at various time intervals post administration, in blood samples drawn from the tip of the tail. Glucose levels were plotted against time post insulin administration (See FIG. 4).

TABLE 4

glucose (mg/dL),
time post administration

| | 0 | 15 | 30 | 45 | 60 | 90 |
|---|---|---|---|---|---|---|
| rat # 1 | 79 | 98 | 85 | 80 | 74 | 70 |
| rat # 2 | 58 | 93 | 91 | 80 | 72 | 69 |
| rat # 3 | 83 | 67 | 80 | 77 | 72 | 72 |
| rat # 4 | 106 | 110 | 107 | 99 | 105 | 93 |
| rat # 5 | 80 | 77 | 50 | 33 | 10 | 10 |
| rat # 6 | 85 | 79 | 55 | 35 | 21 | 33 |
| rat # 7 | 93 | 78 | 53 | 39 | 23 | 31 |

10 = low

As shown in FIG. 4, after the penetrating peptide composition with IBW-002V2 was rectally administered, glucose levels dropped gradually and significantly, in both rats, indicating insulin absorption from the intestine into the blood stream. In contrast, without the peptide a significant drop in glucose levels was noticed only after i.p. administration. No change in blood glucose levels was observed after rectal administration, indicating there was no insulin absorption in these rats.

c) Composition for Translocation of Insulin Using HMIC as the Counter ion and a Penetrating Peptide:

SEQ ID NO: 36 (also called IBW-002V2) and SEQ ID NO: 16 (also called IBW-001) were hydrophobized via acylation of the free amino groups of the two lysine residues at the C-terminus of the penetrating peptides with a myristoyl. Acylation with myristoyl was achieved by incubating the peptide with myristoyl chloride in a molar ratio of 1:10, under basic pH conditions in the presence of appropriate solvents (benzyl benzoate and di-methyl formamide, with 1% bicarbonate). The hydrophobized peptides were then incorporated into the composition, which further contained insulin, and the counter cation 1-hexyl-3-methylimidazolium chloride (HMIC). Additional components of the composition are specified in Table 5.

TABLE 5
A composition for insulin translocation

Hydrophobized Peptide
Insulin
1-hexyl-3-methylimidazolium
chloride (HMIC)
NaOH
Acetic acid
Sodium Acetate
L-arginine
Pluronic F-68
Aprotinin
Solutol HS-15 (SHS)
N-Acetyl Cysteine (NAC)

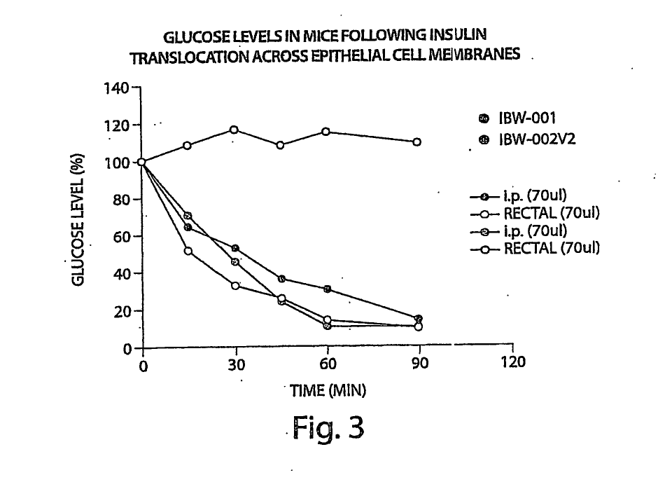

Eight male BALB/c mice, 9-10 weeks old, were deprived of food, 18 hours prior to the experiment. The animals were divided into 4 groups. Each preparation was administered to 2 groups of mice either i.p. (70 ul/mouse, containing 0.2 IU insulin) or rectal (70 ul/mouse, containing 0.2 IU insulin). Blood glucose levels were measured at various time intervals post administration, in blood samples drawn from the tip of the tail. Glucose levels were plotted against time post insulin administration (See FIG. 3).

As can be seen in FIG. 3, after the penetrating peptide composition with IBW-002V2 was administered, glucose levels dropped gradually and significantly, in both groups, indicating insulin absorption from the intestine into the blood stream. In contrast, with the control peptide composition (IBW-001) a significant drop in glucose levels was noticed only after i.p. administration. No change in blood glucose levels was observed after rectal administration, indicating there was no insulin absorption in this group.

In the above examples of compositions for the translocation of insulin across epithelial barriers, blood glucose levels decrease in relation to the amount of insulin absorbed from the intestine into the bloodstream (i.e., in an amount that correlates to the amount of insulin absorbed). Thus, this drug delivery system can replace the need for insulin injections, thereby providing an efficient, safe and convenient route of administration for diabetes patients.

### Example 2

### Utilization of Selective Encapsulation to Enable the Effective Translocation of Heparin Across an Epithelial Barrier

a) Composition for Translocation of Heparin Using BKC as the Counter Ion:

The composition was prepared by lyophilizing heparin and the counter ion benzalkonium chloride (BKC) in a concentration ration of 1:0.5 or 1:1, and then reconstituting them with 2.5% tricaprin in ethanol, and adding a benzyl benzoate: butanol mixture in a ration of 1:11. Additional components of the composition are specified in Table 6.

TABLE 6
Composition for heparin translocation

| Heparin | 10 | mg/ml |
|---|---|---|
| Benzalkonium Chloride (BKC) | 5-10 | mg/ml |
| Tricaprine | 5 | mg/ml |
| Benzyl Benzoate:Butanol 1:11 | 30 | μl/ml |
| Pluronic F-68 | 2% | |
| Aprotinin | 100 | μl/ml |
| Solutol HS-15 (SHS) | 2% | |
| N-Acetyl Cysteine (NAC) | 50 | μg |

In Vivo Experimental Procedure:

Four male CB6/F1 mice, 8-10 weeks old, were deprived of food, 18 hours prior to the experiment The mice were anesthetized by ip. injection of 0.05 ml of a mixture of 0.15 ml xylazine+0.85 ml of ketamin. The composition was then rectally administered to the mice, 100 μl/mouse, using a plastic tip. Penetration was assessed via measurement of clotting time, at different time intervals after heparin administration. Five minutes post administration the tip of the tail was cut and a 50 μl blood sample was drawn into a glass capillary. The capillary was broken at different time intervals, until clot formation was observed. This was repeated at 15,30, and 60 minutes post administration. The animals were subsequently sacrificed. Results are shown below.

TABLE 7

| | | Clotting time, min, measured at follow times | | | | |
|---|---|---|---|---|---|---|
| Mouse # | Heparin:BKC | after injection | | | | |
| (CB6/F1) | Ratio | 0 | 5 min | 15 min | 30 min | 60 min |
| 1 | 1:0.5 | 1.5 | >4 | 11 | 19 | >20 |
| 2 | 1:0.5 | 1 | >5 | >20 | >20 | |
| 3 | 1:1 | 1 | 4 | 12 | >20 | >20 |
| 4 | 1:1 | 1.5 | 5 | 5 | 12 | 19 |

b) Composition for Translocation of Heparin Using BMIC as the Counter Ion and a Penetrating Peptide:

SEQ ID NO: 36 was hydrophobized via acylation of the free amino groups of the two lysine residues at the C-terminus of the penetrating peptide with a myristoyl. Acylation with myristoyl was achieved by incubating the peptide with myristoyl chloride in a molar ratio of 1:10, under basic pH conditions in the presence of appropriate solvents (benzyl benzoate and di-methyl formamide, with 1% bicarbonate). The hydrophobized peptide was then incorporated into the composition, which further contained heparin, and the counter cation 1-butyl-3-methylimidazolium chloride (BMIC). Additional components of the composition are specified in Table 8.

TABLE 8
A composition for heparin translocation

| Hydrophobized SEQ ID NO: 36 | 7.5 | μl/ml |
|---|---|---|
| Heparin | 10 | mg/ml |
| 1-butyl-3-methylimidazolium chloride | 4% | |
| (BMIC) | | |
| N-Methyl Pirolidone (NMP) | 10% | |
| Cremophor EL | 0.37% | |
| Tricaprine | 0.5% | |
| Pluronic F-68 | 2% | |
| rotinin | 20 | μl/ml |
| Solutol HS-15 (SHS) | 2% | |
| N-Acetyl Cysteine (NAC) | 5 | μg/ml |

In Vivo Experimental Procedure:

Four male BALB/c mice, 9-10 weeks old, were deprived of food, 18 hours prior to the experiment. The mice were anesthetized by i.p. injection of 0.0 5ml of a mixture of 0.15 ml xylazine +0.85 ml of ketamin. The composition was then rectally administered to the mice, 100 μl mouse, using a plastic tip covered with a lubricant. Penetration was assessed via measurement of clotting time, at different time intervals after heparin administration. Five minutes post administration the tip of the tail was cut and a 50 μl blood sample was drawn into a glass capillary. The capillary was broken at different time intervals, until clot formation was observed. This was repeated at 15, 30, 60, 90, 120 and 150 minutes post administration. The animals were subsequently sacrificed.

In similar experiments, a control peptide (SEQ ID NO:16), lacking the penetrating peptide-sequence, was similarly hydrophobized and incorporated into the composition shown in Table 8 and then rectally administered to the mice. The average clotting time measured was only slightly elongated compared to that obtained with the full conjugate of the penetrating peptide. Results are shown in Table 9.

TABLE 9

| | Sample | Clotting time, measured at follow times after injection | | | | | | | |
|---|---|---|---|---|---|---|---|---|---|
| Mouse # | injected | 0′ | 5 min | 15 min | 30 min | 60 min | 90 min | 120 min | 150 min |
| 1 | SEQ ID | 1′ | 1′ | 1′ | 2′ | 5′ | 4′ | 2′ | 3′ |
| | NO: 16 | | | | | | | | |
| 2 | SEQ ID | 1.5′ | 1′ | 1′ | 1.5′ | 2.5′ | 5′ | 3′ | 4′ |
| | NO: 36 | | | | | | | | |
| 3 | SEQ ID | 2.5′ | 2′ | 1′ | 3′ | 6′ | 9′* | 8′* | 6′ |
| | NO: 36 | | | | | | | | |
| 4 | SEQ ID | 1.5′ | 1′ | 1.5′ | 1.5′ | 8′* | 9′* | 15′* | 17′* |
| | NO: 36 | | | | | | | | |
| 5 | SEQ ID | 1′ | 2′ | 3′ | 2′ | 9′* | 7′* | 7′* | 9′* |
| | NO: 36 | | | | | | | | |

*indicates appearance of blood clotting, but it did not progress even after several minutes.

In the above examples of compositions for the translocation of heparin across epithelial barriers, clotting time values increase in relation to the amount of heparin absorbed from the intestine into the bloodstream (i.e., in an amount that correlates to the amount of heparin absorbed). Therefore, this drug delivery system can replace the use of heparin injections.

### Example 3

### Utilization of Selective Encapsulation for Mucosal Vaccination

a) Composition for Mucosal Vaccination Using a Counter Ion:

The composition for oral vaccination contains a desired antigenic sequence, i.e. the PA antigen of Anthrax, encapsulated with a counter ion, i.e. benzalkonium chloride, and a hydrophobic agent, ie. tricaprin. Additional possible constituents of the pharmaceutical composition are specified in Table 1. Such a composition can be administered to a subject in need of vaccination.

b) Composition for Mucosal Vaccination Using a Counter Cation and a Penetrating Peptide:

SEQ ID NO: 34 (or any other sequence from SEQ ID NO:22, 30-37) is hydrophobized via acylation of the free amino groups of the two lysine residues at the C-terminus of the penetrating peptide with a fatty acid, i.e., myristoyl. Similarly, any other sequence from SEQ ID NO: 1-15, 24-29 may also be supplemented by extra lysine residues, interspaced by glycine, alanine or serine residues, added at the penetrating peptide C-terminus, and the free amino groups of such lysine residues are acylated with a fatty acid. The hydrophobized peptide is then incorporated into the composition, which further contains a lyophilizate of (1) a desired antigenic sequence, e.g., the PA antigen of Anthrax, (2) an amphipathic counter cation, such as 1-butyl-3-methylimidazolium chloride (BMIC) or 1-hexyl-3-methylimidazolium chloride (HMIC) and (3) phytic acid. Additional constituents are specified in Table 8. Such a pharmaceutical composition can be administered to a subject in need of vaccination.

c) Composition for Mucosal Vaccination Using a Counter Anion and a Penetrating Peptide:

SEQ ID NO: 34 (or any other sequence from SEQ ID NO:22, 30-37) is hydrophobized via acylation of the free amino groups of the two lysine residues at the C-terminus of the penetrating peptide with a fatty acid, i.e., myristoyl. Similarly, any other sequence from SEQ ID NO: 1-15, 24-29 may be also be supplemented by extra lysine residues, interspaced by glycine, alanine or serine residues, added at the penetrating peptide C-terminus, and the free amino groups of such lysine residues are acylated with a fatty acid. The hydrophobized peptide is then incorporated into the composition, which further contains a lyophilizate of (1) a desired antigenic sequence, e.g., the HBs antigen of Hepatitis B, (2) an amphipathic counter anion, such as sodium dodecyl sulfate (SDS) or dioctyl sulfosuccinate (DSS) and (3) phytic acid. Additional constituents are specified in Table 10. Such a pharmaceutical composition can be administered to a subject in need of vaccination.

The composition descnbed above for mucosal vaccination allow for simple and rapid vaccination of large populations in need thereof. Another advantage of this method is the production of high titers of IgA antibodies and the subsequent presence of IgA antibodies in the epithelial mucosa, which are the sites of exposure to antigens.

Efficacy of vaccination can be demonstrated by the measurement of specific antibody titers, IgA in particular, as well as the measurement of immunological response to stimulation, such as for example, via a cutaneous hypersensitivity reaction in response to subcutaneous administration of antigen.

### Example 4

### Utilization of Selective Encapsulation to Enable the Effective Translocation of Aminoglycoside Antibiotics Across an Epithelial Barrier

SEQ ID NO: 34 (or any other sequence from SEQ ID NO:22, 30-37) is hydrophobized via acylation of the free amino groups of the two lysine residues at the C-terminus of the penetrating peptide with a fatty acid, i.e., myristoyl. Similarly, any other sequence from SEQ ID NO: 1-15, 24-29 may be also supplemented by extra lysine residues, interspaced by glycine, alanine or serine residues, added at the penetrating peptide C-terminus, and the free amino groups of such lysine residues are acylated with a fatty acid. The hydrophobized peptide is then incorporated into the penetrating composition, which further contains a lyophilizate of (1) an aminoglycoside antibiotic, i.e., gentamycin, (2) an amphipathic counter anion, such as sodium dodecyl sulfate (SDS) or dioctyl sulfosuccinate (DSS) and (3) phytic acid. Additional constituents are specified in Table 10.

TABLE 10
Additional constituents of the composition

N-Methyl Pirolidone (NMP)
Cremophor EL
Tricaprine
Pluronic F-68
Aprotinin
Solutol HS-15 (SHS)
N-Acetyl Cysteine (NAC)

The composition is administered to test animals, i.e. mice, in two forms: rectally or by injection into an intestinal loop. The experimental procedure involves male BALB/c mice, which are deprived of food, 18 hours prior to the experiment. For intra-intestinal injection the mice are then anesthetized and a 2 cm long incision is made along the center of the abdomen, through the skin and abdominal wall. An intestine loop is gently pulled out through the incision and placed on wet gauze beside the animal. The loop remains intact through the entire procedure and is kept wet during the whole time. The tested compound is injected into the loop, using a 26G needle. For rectal administration, the mice are anesthetized and the composition is then rectally administered to the mice, 100 μl/mouse, using a plastic tip covered with a lubricant.

Penetration is assessed in two methods: (a) direct measurement of antibiotic concentrations in the blood, and (b) measurement of antibacterial activity in serum samples from treated animals.

### Example 5

### Utilization of Selective Encapsulation to Enable the Efficient Translocation of Cationic Antifungal Agents such as Caspofungin Across an Epithelial Barrier

SEQ ID NO: 34 (or any other sequence from SEQ ID NO:22, 30-37) is hydrophobized via acylation of the free amino groups of the two lysine residues at the C-terminus of the penetrating peptide with a fatty acid, ie., myristoyl. Similarly, any other sequence from SEQ ID NO: 1-15, 24-29 may be also supplemented by extra lysine residues, interspaced by glycine, alanine or serine residues, added at the penetrating peptide C-terminus, and the free amino groups of such lysine residues are acylated with a fatty acid. The hydrophobized peptide is then incorporated into the composition, which further contains a lyophilizate of (1) an antifuigal agent, i.e., caspofungin, (2) an amphipathic counter anion, such as sodium dodecyl sulfate (SDS) or dioctyl sulfosuccinate (DSS) and (3) phytic acid. Additional constituents are specified in Table 11.

TABLE 11
Additional constituents of the composition

N-Methyl Pirolidone (NMP)
Cremophor EL
Tricaprine
Pluronic F-68
Aprotinin
Solutol HS-15 (SHS)
N-Acetyl Cysteine (NAC)

The composition is then administered to test animals, i.e., mice, in two forms: rectally or by injection into an intestinal loop. The experimental procedure involves male BALB/c mice, which are deprived of food, 18 hours prior to the experiment. For intra-intestinal injection the mice are then anesthetized and a 2 cm long incision is made along the center of the abdomen, through the skin and abdominal wall. An intestine loop is gently pulled out through the incision and placed on wet gauze beside the animal. The loop remains intact through the entire procedure and is kept wet during the whole time. The tested compound is injected into the loop, using a 26G needle. For rectal administration the mice are anesthetized and the composition is then rectally administered, 100 μl/mouse, using a plastic tip covered with a lubricant.

Penetration is assessed in two methods: (a) direct measurement of caspofungin concentrations in the blood, and (b) measurement of antifungal activity in serum samples from treated animals.

### OTHER EMBODIMENTS

From the foregoing detailed description of the specific embodiments of the invention, it should be apparent that unique methods of translocation across epithelial and endothelial barriers have been described. Although particular embodiments have been disclosed herein in detail, this has been done by way of example for purposes of illustration only, and is not intended to be limiting with respect to the scope of the appended claims that follow. In particular, it is contemplated by the inventor that various substitutions, alterations, and modifications may be made to the invention without departing from the spirit and scope of the invention as defined by the claims. For instance, the choice of the particular type of tissue, or the particular effector to be translocated is believed to be a matter of routine for a person of ordinary skill in the art with knowledge of the embodiments described herein.

1. A composition for transepithelial delivery of at least one effector, comprising a therapeutically effective amount of said at least one effector sequentially coupled with a counter ion to the at least one effector and at least one pharmaceutically acceptable hydrophobic agent, wherein the at least one effector is selectively encapsulated into a complex, and wherein the selectively encapsulated at least one effector is capable of efficiently translocating across a biological barrier.

2. The composition of claim 1, wherein at least 5% of the selectively encapsulated at least one effector is translocated across the biological barrier.

3. The composition of claim 1, wherein at least 10% of the selectively encapsulated at least one effector is translocated across the biological barrier.

4. The composition of claim 1, wherein at least 20% of the selectively encapsulated at least one effector is translocated across the biological barrier.

5. The composition of claim 1, wherein only the selectively encapsulated at least one effector is translocated across the biological barrier, and wherein other molecules concomitantly administered in a non-encapsulated or free form are not translocated across the biological barrier.

6. The composition of claim 1 further comprising a pharmaceutically acceptable excipient, pharmaceutically acceptable carrier, or a combination thereof.

7. The composition of claims 1, wherein said at least one effector is a cationic or an anionic impermeable molecule.

8. The composition of claim 7, wherein said anionic impermeable molecule is a protein, a peptide, a polysaccharide, a nucleic acid or a nucleic acid mimetic.

9. The composition of claim 8, wherein said polysaccharide is aglycosaminoglycan selected from the group consisting of: heparin, heparan sulfate, chondroitin sulfate, dermatan sulfate, hyaluronic acid, and pharmaceutically acceptable salts thereof.

10. The composition of claim 8, wherein the nucleic acid or the nucleic acid mimetic is selected from the group consisting of a DNA, a DNA-mimetic, an RNA, or an RNA-mimetic.

11. The composition of claim 7, wherein said anionic or cationic impermeable molecule is a bioactive molecule selected from the group consisting of: insulin, erythropoietin (EPO), glucagon-like peptide 1 (GLP-1), αMSH, parathyroid hormone (PTH), growth hormone, calcitonin, interleukin-2 (IL-2), αl-antitrypsin, granulocyte/monocyte colony stimulating factor (GM-CSF), granulocyte colony stimulating factor (G-CSF), T20, anti-TNF antibodies, interferon α, interferon β, interferon γ, lutenizing hormone (LH), follicle-stimulating hormone (FSH), enkephalin, dalargin, kyotorphin, basic fibroblast growth factor (bFGF), hirudin, hirulog, lutenizing hormone releasing hormone (LHRH) analog, brain-derived natriuretic peptide (BNP), glatiramer acetate, and neurotrophic factors.

12. The composition of claim 7, wherein said anionic or cationic impermeable molecule is a pharmaceutically active agent selected from the group consisting of: a hormone, a growth factor, a neurotrophic factor, an anticoagulant, a bioactive molecule, a toxin, an antibiotic, an anti-fungal agent, an antipathogenic agent, an antigen, an antibody, an antibody fragment, an immunomodulator, a vitamin, an antineoplastic agent, an enzyme, and a therapeutic agent.

13. The composition of claim 12, wherein said pharmaceutically active agent is selected from the group consisting of vitamin B12, taxol, Caspofungin, or an aminoglycoside antibiotic.

14. The composition of claim 1, wherein said effector further comprises at least one chemical modification.

15. The composition of claim 14, wherein said at least one effector is selected from the group consisting of: insulin, erythropoietin (EPO), glucagon-like peptide 1 (GLP-1), αMSH, parathyroid hormone (PTH), growth hormone, calcitonin, interleukin-2 (IL-2), αl-antitrypsin, granulocyte/monocyte colony stimulating factor (GM-CSF), granulocyte colony stimulating factor (G-CSF), T20, anti-TNF antibodies, interferon α, interferon β, interferon γ, lutenizing hormone (LH), follicle-stimulating hormone (FSH), enkephalin, dalargin, kyotorphin, basic fibroblast growth factor (bFGF), hirudin, hirulog, lutenizing hormone releasing hormone (LHRH) analog, brain-derived natriuretic peptide (BNP), and neurotrophic factors.

16. The composition of claim 14, wherein the chemical modification comprises the attachment of one or more polyethylene glycol residues to the effector.

17. The composition of claim 1, wherein said counter ion is an anionic amphipathic or cationic amphipathic molecule.

18. The composition of claim 17, wherein said anionic amphipathic molecule comprises an organic acid selected from the group consisting of carboxylate, sulfonate and phosphonate anion, and wherein said anionic amphipathic molecule further comprises a hydrophobic moiety.

19. The composition of claim 18, wherein the anionic counter ion is selected from the group consisting of: sodium dodecyl sulphate and dioctyl sulfosuccinate.

20. The composition of claim 17, wherein said cationic amphipathic molecule is a quaternary amine comprising a hydrophobic moiety.

21. The composition of claim 20, wherein said quaternary amine has the general structure of: 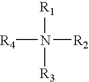
wherein R1, R2, R3 and R4 are alkyl or aryl residues.

22. The composition of claim 21, wherein said quaternary amine is a benzalkonium derivative.

23. The composition of claim 1, wherein said counter ion is an ionic liquid forming cation.

24. The composition of claim 23, wherein said ionic liquid forming cation is selected from the group consisting of imidazolium derivatives, pyridinium derivatives, phosphonium compounds and tetralkylammonium compounds.

25. The composition of claim 24, wherein said imidazolium derivative has the general structure of 1-R1-3-R2-imidazolium, and wherein R1 and R2 are linear or branched alkyls with 1 to 12 carbons.

26. The composition of claim 25, wherein said imidazolium derivative further comprises a halogen or an alkyl group substitution.

27. The composition of claim 24, wherein said imidazolium derivative is selected from the group consisting of: 1-ethyl-3-methylimidazolium; 1-butyl-3-methylimidazolium; 1-hexyl-3-methylimidazolium; 1-methyl-3-octylimidazolium; 1-methyl-3-(3,3,4,4,5,5,6,6,7,7,8,8,8-tridecafluoroctyl)-imidazolium; 1,3-dimethylimidazolium; and 1, 2-dimethyl-3-propylimidazolium.

28. The composition of claim 24, wherein said pyridinium derivative has the general structure of 1-R1-3-R2-pyridinium, where R1is a linear or branched alkyl with 1 to 12 carbons, and R2 is H or a linear or branched alkyl with 1 to 12 carbons.

29. The composition of claim 28, wherein said pyridinium derivative further comprises a halogen or an alkyl group substitution.

30. The composition of claim 24, wherein said pyridinium derivative is selected from the group consisting of 3-methyl-1-propylpyridinium, 1-butyl-3-methylpyridinium, and 1-butyl-4-methylpyridinium.

31. The composition of claim 23, wherein said ionic liquid forming cation is a constituent of a water soluble salt.

32. The composition of claim 1, wherein said hydrophobic agent is selected from the group consisting of aliphatic molecules, cyclic molecules, aromatic molecules, or a combination thereof.

33. The composition of claim 32, wherein said aliphatic hydrophobic agent is selected from the group consisting of: mineral oil, paraffin, fatty, acids, mono-glycerides, di-glycerides, tri-glycerides, ethers, and esters.

34. The composition of claim 33, wherein said tri-glyceride is selected from the group consisting of: long chain triglycerides, medium chain triglycerides, short chain triglycerides, and combinations thereof.

35. The composition of claim 34, wherein said triglyceride is selected from the group consisting of tributyrin, trihexanoin, trioctanoin, and tricaprin (1,2,3-tridecanoyl glycerol).

36. The composition of claim 32, wherein said cyclic hydrophobic agent is selected from the group consisting of: terpenoids, cholesterol, cholesterol derivatives, and cholesterol esters of fatty acids.

37. The composition of claim 32, wherein said aromatic hydrophobic agent is benzyl benzoate.

38. The composition of claim 1, wherein said composition further contains water or an at least partially water soluble solvent selected from the group consisting of: n-butanol, isoamyl (=isopentyl) alcohol, DMF, DMSO, iso-butanol, iso-propanol, propanol, ethanol, ter-butanol, polyols, ethers, amides, esters, and various mixtures thereof.

39. The composition of claim 1, wherein said composition further comprises a protein stabilizer selected from the group consisting of polyanionic molecules, polycationic molecules, uncharged polymers, and combinations thereof.

40. The composition of claim 39, wherein said polyanionic molecule is selected from the group consisting of phytic acid and sucrose octasulfate.

41. The composition of claim 39, wherein said polycationic molecule is a polyamine.

42. The composition of claim 41, wherein said polyamine is spermine.

43. The composition of claim 39, wherein said uncharged polymer is selected from the group consisting of polyvinylpyrrolidone and polyvinyl alcohol.

44. The composition of claim 1, wherein said composition further comprises a penetrating peptide.

45. The composition of claim 44, wherein the penetrating peptide comprises at least one amino acid sequence selected from the group consisting of:
a) (BX)₄Z(BX)₂ZXB;
b) ZBXB₂XBXB₂XBX₃BXB₂X₂B₂;
c) ZBZX₂B₄XB₃ZXB₄Z₂B₂;
d) ZB₉XBX₂B₂ZBXZBX₂;
e) BZB₈XB₉X₂ZXB;
f) B₂ZXZB₅XB₂XB₂X₂BZXB₂;
g) XB₉XBXB₆X₃B;
h) X₂B₃XB₄ZBXB₄XBₙ,XB;
i) XB₂XZBXZB₂ZXBX₃BZXBX₃B;
j) BZXBXZX₂B₄XBX₂B₂XB₄X₂;
k) BZXBXZX₂B₄XBX₂B₂XB₄;
l) B₂XZ₂XB₄XBX₂B₅X₂B₂;
m) B_{q}XₜZBₘX_{q}B₄XBXₙBₘZB₂X₂B₂;
n) B₂ZX₃ZBₘX_{q}B₄XBXₙBₘZB₂X₂B₂;
o) X₃ZB₆XBX₃BZB₂X₂B₂; and
p) at least 12 contiguous amino acids of any of peptides a) through o)
wherein
q is 0 or 1;
m is 1 or 2;
n is 2 or 3;
t is 1 or 2 or 3; and
X is any amino acid;
B is a hydrophobic amino acid; and
Z is a charged amino acid;
wherein said penetrating peptide is capable of translocating across a biological barrier.

46. The composition of claim 45, wherein the penetrating peptide comprises an amino acid sequence selected from the group consisting of:
a) SEQ ID NOS:1, 2, 3, 4, 5, 6, 7, 8, 9, 10, 11, 12, 13, 14, 15, 24, 25, 26, 27, 28 and 29;
b) a variant of an amino acid sequence selected from the group consisting of SEQ ID NOS: 1, 2, 3, 4, 5, 6, 7, 8, 9, 10, 11, 12, 13, 14, 15, 24, 25, 26, 27, 28 and 29, wherein one or more amino acid residues in said variant differs from the amino acid sequence of said penetrating peptide, provided that said variant differs in no more than 15% of amino acid residues from said amino acid sequence;
c) a fragment of an amino acid sequence selected from the group consisting of SEQ ID NOS: 1, 2, 3, 4, 5, 6, 7, 8, 9, 10, 11, 12, 13, 14, 15, 24, 25, 26, 27, 28 and 29; and
d) a peptide comprising at least 12 contiguous amino acids of any of the peptides selected from the group consisting of SEQ ID NOS: 1, 2, 3, 4, 5, 6, 7, 8, 9, 10, 11, 12, 13, 14, 15,24,25,26,27,28 and 29.

47. The composition of claim 46, wherein the fragment is at least 10 amino acids in length.

48. The composition of claim 46, wherein the amino acid sequence of said variant comprises a conservative amino acid substitution.

49. The composition of claim 46, wherein the amino acid sequence of said variant comprises a non-conservative amino acid substitution.

50. The composition of claim 46, wherein the penetrating peptide is further modified, via one or more peptidic bonds, to enable protection from gastrointestinal proteolysis.

51. The composition of claim 50, wherein one or more amino acid residues in said variant is replaced by a non-naturally occurring amino acid, selected from the group consisting of: D-amino acids, norleucine, norvaline, homocysteine, homoserine, ethionine, and compounds derivatized with an amino-terminal blocking group selected from the group consistingof t-butyloxycarbonyl, acetyl, methyl, succinyl, methoxysuccinyl, suberyl, adipyl, azelayl, dansyl, benzyloxycarbonyl,fluorenylmethoxycarbonyl, methoxyaselayl, methoxyadipyl, methoxysuberyl, and a 2,3-dinitrophenyl group.

52. The composition of claim 50, wherein one or more peptide bonds have been replaced with an alternative type of covalent bond to form a peptide mimetic.

53. The composition of claim 45, wherein the penetrating peptide is the peptide of SEQ ID NO: 3 or at least 12 contiguous amino acids thereof.

54. The composition of claim 45, wherein the penetrating peptide is the peptide of SEQ ID NO: 8 or at least 12 contiguous amino acids thereof.

55. The composition of claim 45, wherein the penetrating peptide is the peptide of SEQ ID NO: 9 or at least 12 contiguous amino acids thereof.

56. The composition of claim 45, wherein the penetrating peptide is the peptide of SEQ ID NO: 12 or at least 12 contiguous amino acids thereof.

57. The composition of claim 45, wherein penetrating peptide is the peptide of SEQ ID NO: 24 or at least 12 contiguous amino acids thereof.

58. The composition of claim 45, wherein the penetrating peptide is less than 30 amino acids long.

59. The composition of claim 45, wherein the penetrating peptide is less than 25 amino acids long.

60. The composition of claim 45, wherein the penetrating peptide is less than 20 amino acids long.

61. The composition of claim 45, wherein said penetrating peptide further contains lysine residues, interspaced by glycine, alanine or serine residues, added at the C-terminus of the penetrating peptide, and wherein the free amino groups of said lysine residues are acylated.

62. The composition of claim 61, wherein acylation utilizes long-chain fatty acids selected from the group consisting of: stearoyl, palmitoyl, oleyl, ricinoleyl, lauroyl and myristoyl.

63. The composition of claim 61, wherein the amino acid sequence of the penetrating peptide is selected from the group consisting of:
a) SEQ ID NOS: 22, 30, 31, 32, 33, 34, 35, 36, and 37;
b) a variant of an amino acid sequence selected from the group consisting of SEQ ID NOS: 22, 30, 31, 32, 33, 34, 35, 36, and 37, wherein one or more amino acid residues in said variant differs from the amino acid sequence of said penetrating peptide, provided that said variant differs in no more than 15% of amino acid residues from said amino acid sequence;
c) a fragment of an amino acid sequence selected from the group consisting of SEQ ID NOS: 22, 30, 31, 32, 33, 34, 35, 36, and 37; and
d) a peptide comprising at least 12 contiguous amino acids of any of the peptides selected from the group consisting of SEQ ID NOS: 22, 30, 31, 32, 33, 34, 35, 36, and 37.

64. The composition of claims 44 or 45, wherein said penetrating peptide further comprises a chemical modification.

65. The composition of claim 64, wherein the chemical modification comprises the attachment of one or more polyethylene glycol residues to the penetrating peptide.

66. The composition of claim 1, wherein said composition further comprises a surface active agent selected from the group consisting of an ionic detergent, a non-ionic detergent, or a combination thereof.

67. The composition of claim 66, wherein said ionic detergent is selected from the group consisting of fatty acid salts, lecithin, bile salts, and combinations thereof.

68. The composition of claim 66, wherein said non-ionic detergent is selected from the group consisting of: a poloxamer, Solutol HS15, Cremophore, a polyethylene glycol fatty alcohol ether, sorbitan fatty acid esters, and combinations thereof.

69. The composition of claim 68, wherein said sorbitan fatty acid ester is selected from the group consisting of sorbitan monolaurate, sorbitan monooleate, sorbitan monopalmitate, and combinations thereof.

70. The composition of claim 1, further comprising at least one protective agent.

71. The composition of claim 70, wherein said protective agent is a protease inhibitor selected from the group consisting of: aprotinin, Bowman-Birk inhibitor, soybean trypsin inhibitor, chicken ovomucoid, chicken ovoinhibitor, human pancreatic trypsin inhibitor, camostate mesilate, flavonoid inhibitors, antipain, leupeptin, p-aminobenzamidine, AEBSF, TLCK, APMSF, DFP, PMSF, poly(acrylate) derivatives, chymostatin, benzyloxycarbonyl-Pro-Phe-CHO, FK-448, sugar biphenylboronic acids complexes, β-phenylpropionate, elastatinal, methoxysuccinyl-Ala-Ala-Pro-Val-chloromethylketone (MPCMK), EDTA, chitosan-EDTA conjugates, amino acids, di-peptides, tripeptides, amastatin, bestatin, puromycin, bacitracin, phosphinic acid dipeptide analogues, α-aminoboronic acid derivatives, Na-glycocholate, 1,10-phenantroline, acivicin, L-serine-borate, thiorphan, phosphoramidon, and combinations thereof.

72. The composition of claim 1, wherein the composition further comprises a mixture of at least two substances selected from the group consisting of a non-ionic detergent, an ionic detergent, a protease inhibitor, a sulfohydryl group status modifying agent, and an antioxidant.

73. The composition of claim 72, wherein the non-ionic detergent is a poloxamer, cremophore, a polyethylene glycol fatty alcohol ether, a sorbitan fatty acid ester, or Solutol HS15.

74. The composition of claim 72, wherein the ionic detergent is a fatty acid salt.

75. The composition of claim 72, wherein the protease inhibitor is selected from the group consisting of aprotinin, Bowman-Birk inhibitor, soybean trypsin inhibitor, chicken ovomucoid, chicken ovoinhibitor, human pancreatic trypsin inhibitor, camostate mesilate, flavonoid inhibitors, antipain, leupeptin, p-aminobenzamidine, AEBSF, TLCK, APMSF, DFP, PMSF, poly(acrylate) derivatives, chymostatin, benzyloxycarbonyl-Pro-Phe-CHO, FK-448, sugar biphenylboronic acids complexes, β-phenylpropionate, elastatinal, methoxysuccinyl-Ala-Ala-Pro-Val-chloromethylketone (MPCMK), EDTA, chitosan-EDTA conjugates, amino acids, di-peptides, tripeptides, amastatin, bestatin, puromycin, bacitracin, phosphinic acid dipeptide analogues, α-aminoboronic acid derivatives, Na-glycocholate, 1,10-phenantroline, acivicin, L-serine-borate, thiorphan, phosphoramidon, and combinations thereof.

76. The composition of claim 72, wherein the sulfohydryl group status modifying agent is selected from the group consisting of NAC and Diamide.

77. The composition of claim 72, wherein the antioxidant is selected from the group consisting of tocopherol, deteroxime mesylate, methyl paraben, ethyl paraben, ascorbic acid, and combinations thereof.

78. The composition of claim 1, wherein said composition is contained within a capsule.

79. The composition of claim 1, wherein said composition is in the form of a tablet, an emulsion, a suspension, a cream, an ointment, an aqueous dispersion, a suppository, or a nasal spray.

80. The composition claim 1, wherein said composition is enteric-coated.

81. A kit comprising, in one or more containers, a therapeutically or prophylactically effective amount of the composition of claim 1.

82. The composition of claim 44, wherein the peptide is derived from an integral membrane protein.

83. The composition of claim 44, wherein the peptide is derived from a bacterial toxin.

84. The composition of claim 44, wherein the peptide is derived from an extracellular protein.

85. An isolated peptide comprising an amino acid sequence selected from the group consisting of SEQ ID NOS: 1-8, 10-15, and 25-29, wherein said peptide is derived from a bacterial protein, and wherein said peptide is characterized by the ability to penetrate biological barriers in vivo.

86. An isolated peptide comprising an amino acid sequence selected from the group consisting of SEQ ID NOS: 9 and 24, wherein said peptide is derived from a human neurokinin receptor, and wherein said peptide is characterized by the ability to penetrate biological barriers in vivo.

87. A method of producing the composition of claim 44, said method comprising coupling a therapeutically effective amount of the at least one effector with a penetrating peptide and a counter ion to the at least one effector.

88. A method for producing the composition of claim 44, the method comprising synthesizing the penetrating peptide using solid-phase synthesis, and coupling the penetrating peptide to at least one effector and a counter ion to the effector.

89. The method of claims 87 or 88, wherein the coupling of said at least one effector and said penetrating peptide is achieved by a non-covalent bond.

90. The method of claim 89, wherein the non-covalent bond is achieved by an attachment of a hydrophobic moiety to the penetrating peptide, wherein the hydrophobic moiety enables the penetrating peptide to be incorporated at the interface of a hydrophobic vesicle in which the at least one effector is contained.

91. A method of translocating at least one effector across a biological barrier, said method comprising:
a) coupling said at least one effector with a counter ion and a penetrating peptide to produce the hydrophobic composition of claim 44; and
b) introducing said hydrophobic composition to the biological barrier.

92. A method for producing the composition of claim 1, the method comprising lyophilizing the effector and the counter ion by any suitable means, and subsequently reconstituting the lyophilized materials in an aqueous or organic solvent that is partially soluble in water, or a combination thereof, thereby producing the composition.

93. The method of claim 92, wherein the lyophilizing step alternatively comprises optionally lyophilizing the effector and the counter ion with a protein stabilizer, a penetrating peptide, or any other constituent of a pharmaceutical excipient or carrier.

94. A method of translocating at least one effector across a biological barrier comprising introducing the composition claim 1 to a biological barrier and allowing the at least one effector to translocate across said biological barrier.

95. The method of claim 94, wherein the translocation across a biological barrier occurs within a tissue selected from the group consisting of: epithelial cells and endothelial cells.

96. The method of claim 94, wherein said biological barrier is selected from the group consisting of: tight junctions and plasma membranes.

97. The method of claim 94, wherein said biological barrier is selected from the group consisting of the gastro-intestinal mucosa and the blood blain barrier.

98. A method of treating or preventing a disease or pathological condition, said method comprising administering to a subject in which such treatment or prevention is desired, the composition claim 1, in an amount sufficient to treat or prevent said disease or said pathological condition in said subject.

99. The method of claim 98, wherein said disease or said pathological condition is selected from the group consisting of: endocrine disorders, diabetes, infertility, hormone deficiencies, osteoporosis, ophthalmological disorders, neurodegenerative disorders, Alzheimer's disease, dementia, Parkinson's disease, multiple sclerosis, Huntington's disease, cardiovascular disorders, atherosclerosis, hyper-coagulable states, hypo-coagulable states, coronary disease, cerebrovascular events, metabolic disorders, obesity, vitamin deficiencies, renal disorders, renal failure, haematological disorders, anemia of different entities, immunologic and rheumatologic disorders, autoimmune diseases, immune deficiencies, infectious diseases, viral infections, bacterial infections, ftungal infections, parasitic infections, neoplastic diseases, multifactorial disorders, impotence, chronic pain, depression, different fibrosis states, and short stature.

100. A method of mucosal vaccination, the method comprising administering to a subject in need of vaccination the composition of claim 1, wherein the at least one effector comprises an antigen to which vaccination is desirable.

101. The method of claim 100, wherein the antigen to which vaccination is desired is selected from the group consisting of PA for use in a vaccine against Anthrax and HBs for use in a vaccine against Hepatitis B.

102. The method of any one of claims 98-101, wherein the composition is administered via a route of administration selected from the group consisting of: oral, nasal, transdermal, buccal, sublingual, anal, rectal, bronchial, pulmonary, intraorbital, parenteral, and topical.
